(19) United States
(12) Patent Application Publication
(10) Pub. No.: US 2026/0241264 A1
(43) Pub. Date: Aug. 20, 2026

(54) METHODS, SYSTEMS, AND DEVICES FOR CAPTURING VIDEO CONTENT ASSOCIATED WITH PERFORMING AN ATHLETIC SKILL AND DETERMINING BIOMECHANIC ADJUSTMENTS FOR PERFORMING THE ATHLETIC SKILL

(71) Applicant: ATHLETIQ, LLC, Lubbock, TX (US)

(72) Inventors: Matthew Mooney, Wauconda, IL (US); Matthew Thomas Mitchell, Crown Point, IN (US)

(73) Assignee: ATHLETIQ, LLC, Lubbock, TX (US)

(21) Appl. No.: 19/536,862

(22) Filed: Feb. 11, 2026

Related U.S. Application Data
(63) Continuation-in-part of application No. 19/241,708, filed on Jun. 18, 2025.
Application No. 19/241,708 is a continuation-in-part of application No. PCT/US2023/084860, filed on Dec. 19, 2023.
Application No. PCT/US2023/084860 is a continuation-in-part of application No. PCT/US2024/041481, filed on Aug. 8, 2024.
(60) Provisional application No. 63/476,294, filed on Dec. 20, 2022.
(60) Provisional application No. 63/518,480, filed on Aug. 9, 2023.

(51) Int. Cl. A63B 71/06

(52) U.S. Cl. CPC A63B 71/0669; A63B 2220/806

(57) ABSTRACT

Aspects of the subject disclosure may include, for example, obtaining current video content of a player repeatedly performing a physical skill, analyzing the current video content based on previous video content, the previous video content comprises other video content of the player repeatedly performing the physical skill, determining biomechanic metrics of the player performing the physical skill based on the analysis, and determining each biomechanic metric of a portion of the biomechanic metrics does not satisfy a respective biomechanic metric success rate. Further embodiments include generating a first image of the player performing the physical skill from the current video content, generating a second image of the player performing the physical skill from the previous video content, and presenting the first image and the second image simultaneously and indicating the portion of the biomechanics that did not satisfy the respective biomechanic metric success rate. Other embodiments are disclosed.

### CROSS-REFERENCE TO RELATED APPLICATION(S)

The present application claims priority to and is a continuation in part of U.S. patent application Ser. No. 19/241,708, filed Jun. 18, 2025, which is a continuation in part of Patent Cooperation Treaty Application No. PCT/US2023/084860, filed Dec. 19, 2023, which claims the benefit of priority to U.S. Provisional Application No. 63/476,294, filed Dec. 20, 2022. Further, U.S. patent application Ser. No. 19/241,708, filed Jun. 18, 2025, claims priority to and is a continuation in part of Patent Cooperation Treaty Application No. PCT/US2024/041481, filed Aug. 8, 2024, which claims the benefit of priority to U.S. Provisional Application No. 63/518,480, filed Aug. 9, 2023. All sections of the aforementioned application(s) and/or patent(s) are incorporated herein by reference in their entirety.

### FIELD OF THE DISCLOSURE

The subject disclosure relates to methods, systems, and devices for capturing video content associated with performing an athletic skill and determining biomechanic adjustments for performing the athletic skill based on correlation of biomechanics to success rates.

### BACKGROUND

In athletic performance and analytics, the ability to accurately assess and improve an athlete's skills can be challenging. Traditional methods of evaluating athletic performance, particularly in sports like basketball, often rely on subjective observations and manual tracking of metrics such as balance, alignment, movement, footwork, and other biomechanics. These methods can be inconsistent and fail to provide the comprehensive feedback necessary for athletes to refine their skills effectively. Moreover, existing technologies that attempt to automate this process often lack the precision and adaptability required to cater to the specific biomechanics of individual athletes. Further, the current state of the art falls short in providing real-time, actionable insights that can be tailored to the specific needs of each athlete. This limitation hinders athletes from maximizing their training sessions or gameplay and achieving optimal performance.

### BRIEF DESCRIPTION OF THE DRAWINGS

Reference will now be made to the accompanying drawings, which are not necessarily drawn to scale, and wherein:

FIG. 1, FIG. 2A, FIG. 2B, FIG. 3, and FIG. 4 are block diagrams illustrating exemplary, non-limiting embodiments of a system to determine the biomechanics of a player to perform a skill in accordance with various aspects described herein.

FIG. 5 depicts an illustrative embodiment of a method in accordance with various aspects described herein.

FIG. 6A, FIG, 6B, FIG. 7, FIG. 8, and FIG. 9 are block diagrams illustrating exemplary, non-limiting embodiments of a system to determine the biomechanics of a player to perform a skill in accordance with various aspects described herein.

FIG. 10 is a block diagram of an example, non-limiting embodiment of a computing environment in accordance with various aspects described herein.

FIG. 11A, FIG. 11B, FIG. 11C, FIG. 11D, FIG. 11E, FIG. 11F, FIG. 11G, FIG. 11H, and FIG. 11I are block diagrams illustrating exemplary, non-limiting embodiments of a system to determine the biomechanics of a player to perform a skill in accordance with various aspects described herein.

FIG. 12A, FIG. 12B, FIG. 12C, FIG. 12D, FIG. 12E, and FIG. 13A depicts an illustrative embodiment of a method in accordance with various aspects described herein.

### DETAILED DESCRIPTION

The subject disclosure describes, among other things, illustrative embodiments for obtaining video content of a player repeatedly performing a physical skill during a first time period resulting in current video content, analyzing the current video content utilizing an AI software application based on previous video content resulting in an analysis, where the previous video content comprises other video content of the player repeatedly performing the physical skill during a second time period, and where the second time period is prior to the first time period. A group of biomechanic metrics can then be determined, which are associated with the player performing the physical skill based on the analysis utilizing the AI software application resulting in a first determination. Further embodiments include, based on the first determination, determining each biomechanic metric of a portion of the group of biomechanic metrics does not satisfy a respective biomechanic metric success rate from a group of respective biomechanic metric success rates resulting in a second determination, based on the second determination, generating a first image of the player performing the physical skill from the current video content; based on the second determination, generating a second image of the player performing the physical skill from the previous video content, and presenting the first image and the second image simultaneously on the device with an indication of the portion of the group of biomechanics that did not satisfy the respective biomechanic metric success rate. Some embodiments determine the success rate of each biomechanic metric, then comparing each biomechanic metric to the player's average success rate for the performing the physical skill then providing feedback/actionable insights based on the difference between each biomechanic metric's success rate and the player's overall success rate in performing the physical skill. Further embodiments indicate which biomechanics led to higher success rates, making the player aware of the difference and providing actionable insights for improvement. Other embodiments are described in the subject disclosure.

One or more aspects of the subject disclosure include a device. comprising a processing system including a processor, and a memory that stores executable instructions that, when executed by the processing system, facilitate performance of operations. The operations can comprise obtaining video content of a player repeatedly performing a physical skill during a first time period resulting in current video content, analyzing the current video content utilizing an AI software application based on previous video content resulting in an analysis, the previous video content comprises other video content of the player repeatedly performing the physical skill during a second time period, and the second time period is prior to the first time period. Further operations can comprise determining a group of biomechanic metrics associated with the player performing the physical skill based on the analysis utilizing the AI software application resulting in a first determination, based on the first determination, determining each biomechanic metric of a portion of the group of biomechanic metrics does not satisfy a respective biomechanic metric success rate from a group of respective biomechanic metric success rates resulting in a second determination, based on the second determination, generating a first image of the player performing the physical skill from the current video content, based on the second determination, generating a second image of the player performing the physical skill from the previous video content, and presenting the first image and the second image simultaneously on the device with an indication of the portion of the group of biomechanics that did not satisfy the respective biomechanic metric success rate.

One or more aspects of the subject disclosure include a non-transitory, machine-readable storage device, comprising executable instructions that, when executed by a processing system including a processor, facilitate performance of operations. The operations can comprise obtaining video content of a player repeatedly performing a physical skill during a first time period resulting in current video content, analyzing the current video content utilizing an AI software application based on previous video content resulting in an analysis, the previous video content comprises other video content of the player repeatedly performing the physical skill during a second time period, the second time period is prior to the first time period, and determining a group of biomechanic metrics associated with the player performing the physical skill based on the analysis utilizing the AI software application resulting in a first determination. Further operations can comprise based on the first determination, determining each biomechanic metric of a portion of the group of biomechanic metrics does not satisfy a respective biomechanic metric success rate from a group of respective biomechanic metric success rates resulting in a second determination, and based on the second determination, generating a first image of the player performing the physical skill from the current video content. Additional operations can comprise based on the second determination, generating a second image of the player performing the physical skill from the previous video content, and providing the first image and the second image to a computing device over a communication network, the computing device presents the first image and the second image simultaneously on the computing device with an indication of the portion of the group of biomechanics that did not satisfy the respective biomechanic metric success rate.

One or more aspects of the subject disclosure include a method. The method can comprise obtaining, by a processing system including a processor, video content of a player repeatedly performing a physical skill during a first time period resulting in current video content utilizing a group of camera sensors, each camera sensor of the group of camera sensors are oriented in a respective first position, and determining, by the processing system, that a group of biomechanic metrics cannot be determined from a portion of the current video content utilizing an AI software application resulting in a first determination. Further, the method can comprise based on the first determination, adjusting, by the processing system, a portion of the group of camera sensors from the respective first position to a respective second position, analyzing, by the processing system, the current video content utilizing the AI software application based on previous video content resulting in an analysis, the previous video content comprises other video content of the player repeatedly performing the physical skill during a second time period, the second time period is prior to the first time period, and determining, by the processing system, the group of biomechanic metrics associated with the player performing the physical skill based on the analysis utilizing the AI software application resulting in a second determination. In addition, the method can comprise based on the second determination, determining, by the processing system, each biomechanic metric of a portion of the group of biomechanic metrics does not satisfy a respective biomechanic metric success rate from a group of respective biomechanic metric success rates resulting in a third determination, based on the third determination, generating, by the processing system, a first image of the player performing the physical skill from the current video content, based on the third determination, generating, by the processing system, a second image of the player performing the physical skill from the previous video content, and presenting, by the processing system, the first image and the second image simultaneously on the processing system with an indication of the portion of the group of biomechanics that did not satisfy the respective biomechanic metric success rate.

In the area of athletic performance, repeatedly and consistently performing an athletic skill is important for success. However, achieving and maintaining such proficiency when repeatedly performing the athletic skill requires regular assessment and improvement of various biomechanics involved in performing the skill. Players often face challenges in improving their practice sessions and gameplay to hone these skills effectively. Existing technologies in the field of athletic performance analytics have attempted to address these challenges by providing feedback on a player's biomechanics. These technologies utilize AI or machine learning (ML) models that aggregate data from a wide range of players to predict athletic skill success and suggest adjustments to align with purported “ideal” biomechanics to perform the athletic skill. However, these approaches often fail to account for the specific biomechanics of individual players, and/or other characteristics of the player (including physical characteristics such as height, weight, lengths of arms, size of hands/feet, etc.) and/or associated with movement by the player (including speed, quickness, jumping ability, and so forth) that may influence biomechanics of the individual players, leading to recommendations that may not be optimal for all athletes. Additionally, the reliance on generalized data can result in feedback that does not accurately reflect the player's personal strengths and weaknesses, limiting their effectiveness. Embodiments described herein, aim to address these limitations by employing AI/ML to analyze a player's own shooting data, focusing on individual biomechanics and actual performance-of-skill success rates. By tracking and correlating various biomechanic metrics with success rates, one or more embodiments determine effective biomechanic adjustments in performing the athletic skill for the individual player. This personalized approach enables athletes to make informed decisions about performing the athletic skill, enhancing their performance by leveraging their personal strengths and providing insights based on data.

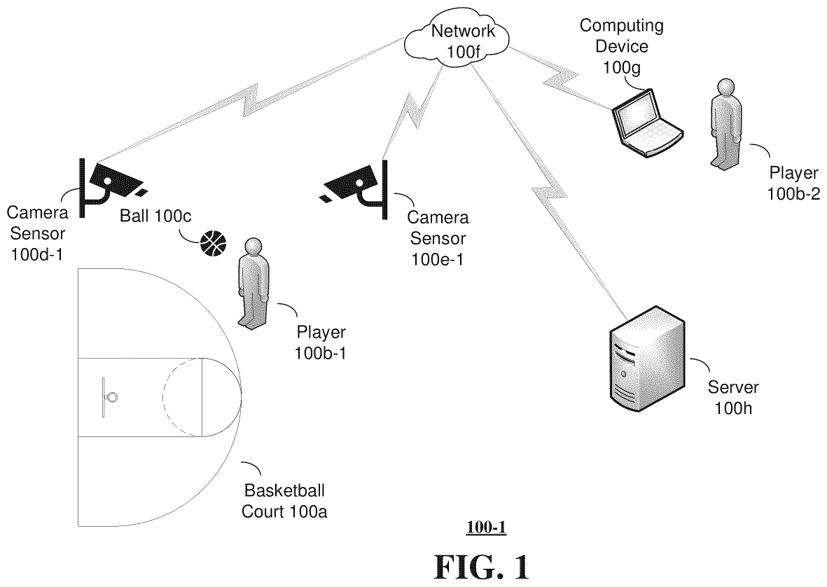

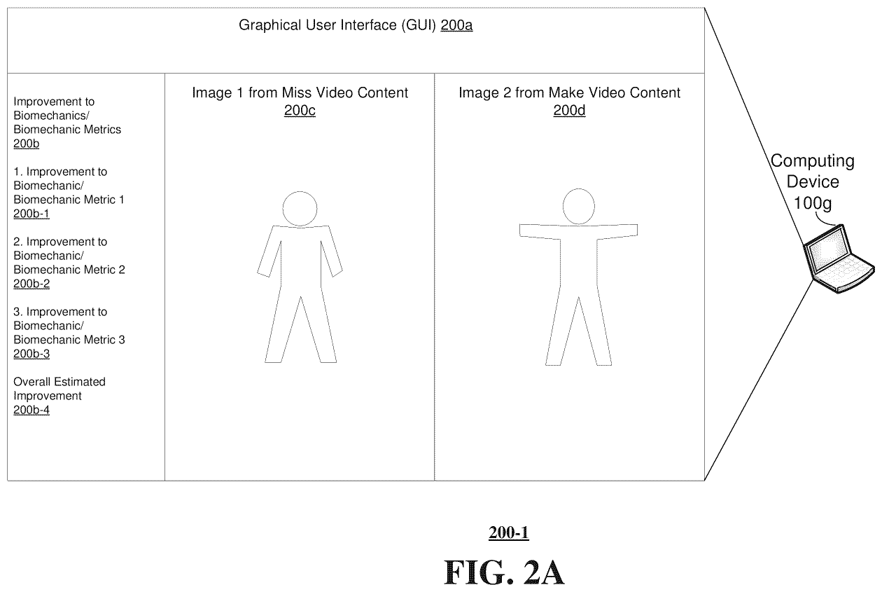

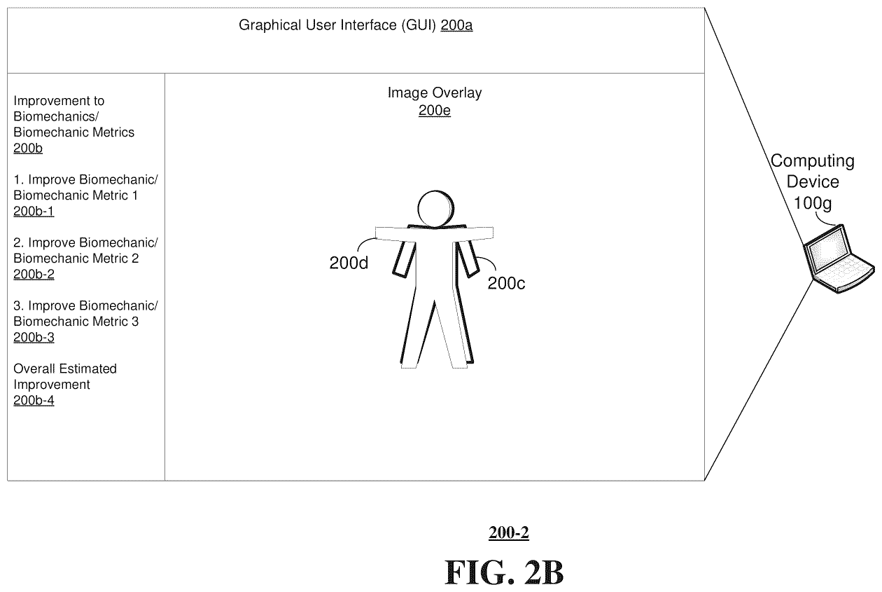

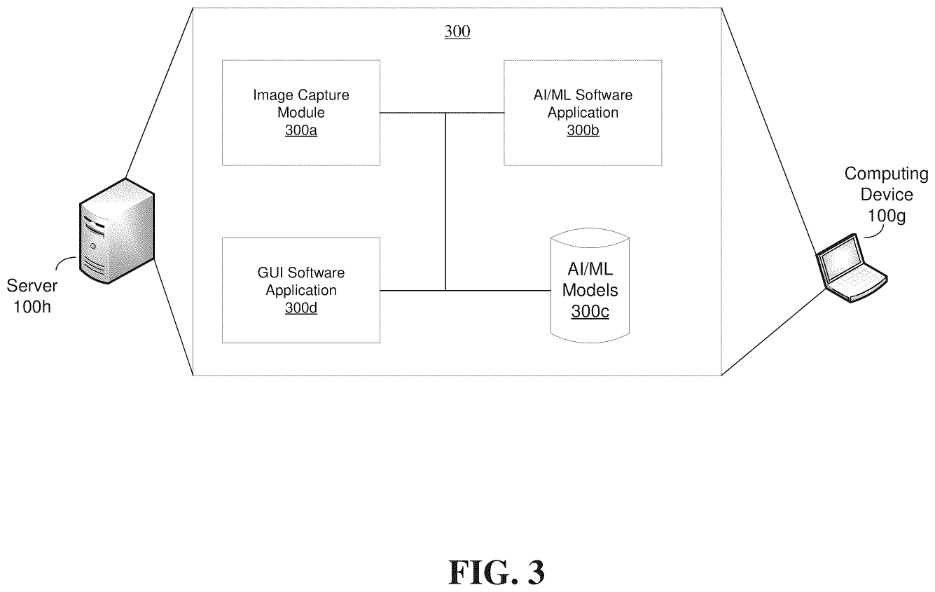

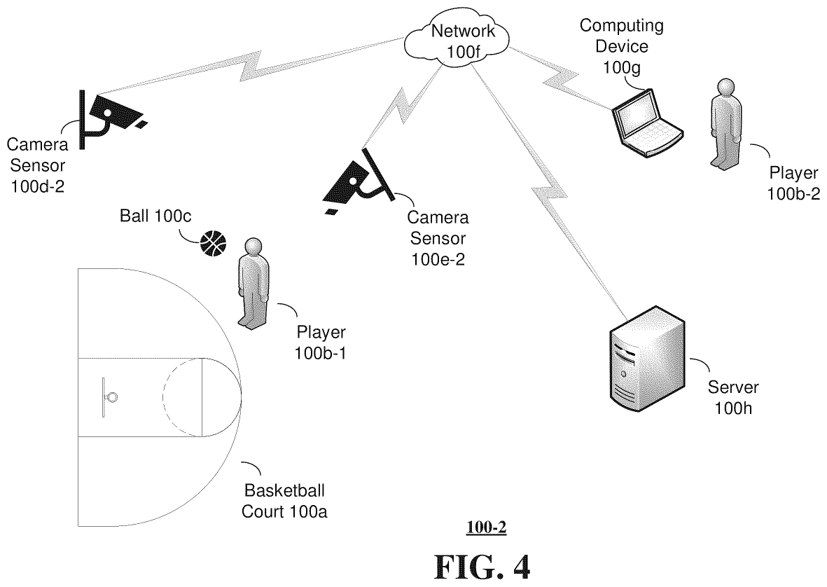

FIGS. 1-4 are block diagrams illustrating exemplary, non-limiting embodiments of a system to determine the biomechanics adjustment of a player to perform a skill in accordance with various aspects described herein. Referring to FIG. 1, in one or more embodiments, the system 100-1 comprises a basketball court 100a, where a player 100b-1, is engaged in shooting practice using basketball 100c. Further, the system 100 can include camera sensor 100d-1 and camera sensor 100e-1, which are strategically positioned around the basketball court 100a. In addition, the system 100 comprises a computing device 100g and a server 100h. Each of camera sensor 100d-1, camera sensor 100e-1, computing device 100g, and server 100h are communicatively coupled to one another via network 100f. Any number of camera sensors and any configuration can be utilized to facilitate of the operation of system 100-1.

In one or more embodiments, each of camera sensor 100d-1 and camera sensor 100e-1 can be a camera that can be remotely controlled by computing device 100g and/or server 100h. In further embodiments, each of camera sensor 100d-1 and camera sensor 100e-1 can include a smartphone camera, mobile device camera, a tablet camera, a digital camera, etc. In additional embodiments, position of each of camera sensor 100d-1 and camera sensor 100e-1 can be remotely controlled by computing device 100g and/or server 100h. In some embodiments, each of camera sensor 100d-1 and camera sensor 100 e-1 can be moved laterally, radially, up and down, back and forth, and in any 360-degree direction. In some embodiments, each of camera sensor 100d-1 and camera sensor 100e-1 can be integrated in a system that can include computing device 100g and/or server 100h.

In one or more embodiments, network 100f can comprise one or more wired communication networks, one or more wireless communication networks, or a combination thereof. Further, computing device 100g can comprise a laptop computer, a desktop computer, a tablet computer, a smartphone, mobile phone, mobile device, a wearable device, a smartwatch or any other computing device, or combination thereof. In addition, server 100h can comprise one or more servers in one location, one or more servers spanning multiple locations, one or more virtual servers in one location, one or more virtual servers spanning multiple locations, one or more cloud servers, or combination thereof.

In one or more embodiments, player 100b-1 can perform an athletic skill such as shooting a basketball 100c on basketball court 100a. Further, each of camera sensor 100d-1 and camera sensor 100e-1 can capture real-time video content (e.g., images, video, video clips, etc.) of the movements of player 100b-1 and their biomechanics while performing their athletic skill (e.g., shooting basketball 100c). Biomechanics can include, but not limited to the mechanical, muscular, or movement associated with performing the athletic skill including a movement pattern associated with performing the athletic skill as well as the movement associated with the kinetic chain in performing the athletic skill. In some embodiments, each of camera sensor 100d-1 and camera sensor 100e-1 can be configured to maintain a fixed perspective relative to player 100b-1 and the basketball hoop of basketball court 100a, ensuring consistent pixel mapping for accurate analysis. The basketball 100c can be tracked, individually or collectively, by each of camera sensor 100d-1 and camera sensor 100e-1. In other embodiments, each of camera sensor 100d-1 and camera sensor 100e-1 can be mobile and move to track the movement of the basketball 100c during the shot from player 100b-1 to the basketball hoop and/or the movement of player 100b-1 themself. Further, each of camera sensor 100d-1 and camera sensor 100e-1 can record video content associated with player 100b-1 repeatedly performing the athletic skill (e.g., shooting basketball 100c) and determine whether the player successfully performed the athletic skill or not (e.g., making or missing the basketball shot) such that further components of system 100 can analyze each repetition of the athletic skill and interaction of the player's movements while performing each repetition.

In one or more embodiments, each of camera sensor 100d-1 and camera sensor 100e-1 can record video content of player 100b-1 while practicing performing of the athletic skill (e.g., shooting basketball 100c from 3-point range) during a practice session. A respective portion of this practice video content can be transmitted by each of camera sensor 100d-1 and camera sensor 100e-1 to computing device 100g and/or server 100h over network 100f. Further, an AI/ML software application on either computing device 100g or server 100h, described herein, can be configured to analyze the practice video content to determine a group of biomechanics and a group of biomechanic metrics for player 100b-1 associated with the repeated performance of the athletic skill (e.g., shooting basketball 100c). Further, the AI software application can determine a portion of the group of biomechanic metrics that provide success in performance of the athletic skill over an overall success rate (e.g., shooting basketball from 3-point range over 50% overall shooting percentage and/or the shooting percentage for each shot type) for particular player 100b-1. Moreover, player 100b-2 can review the portion of the group of biomechanic metrics at computing device 100g to succeed in performing the athletic skill (e.g., shooting basketball 100c from 3-point range) during future gameplay.

In one or more embodiments, each of camera sensor 100d-1 and camera sensor 100e-1 can record video content of player 100b-1 while performing the athletic skill (e.g., shooting basketball 100c from 3-point range) during gameplay (subsequent to a training session). A respective portion of this game video content can be transmitted by each of camera sensor 100d-1 and camera sensor 100e-1 to computing device 100g and/or server 100h over network 100f. Further, the AI/ML software application on either computing device 100g or server 100h, described herein, can be configured to analyze the game video content to determine a group of biomechanic metrics for player 100b-1 associated with the repeated performance of the athletic skill (e.g., shooting basketball 100c) during gameplay. In one or more embodiments, the capturing of data and analysis as described herein, can be performed by system 100-1 in real-time or near-real-time (e.g., within a particular time threshold that allows for timely feedback to a player, such as right after a timeout which may be within less than a minute of the analyzed gameplay). Further, the AI/ML software application can determine a portion of the group of biomechanics and their associated biomechanic metrics that can be improved by particular player 100b-1 to be more successful in performing the athletic skill (e.g., shooting basketball from 3-point range). Moreover, player 100b-2 can review the portion of the group of biomechanics and their associated biomechanic metrics that they need to improve at computing device 100g during a break of gameplay (e.g. timeout, halftime, etc.). It should be understood that while system 100-1 is described with respect to the sport of basketball, the components and functionality associated with the system can be applied to various sports or activities where biometrics can be analyzed to improve performance.

Referring to FIG. 2A, in one or more embodiments, system 200-1 comprises a graphical user interface (GUI) 200a that can be displayed or presented on computing device 100g. In some embodiments, the GUI 200a (including any indications and images/video clips) therein can be generated by computing device 100g utilizing an AI/ML software application. In other embodiments, the information shown on GUI 200a (including any indications and images/video clips) therein can be generated by server 100h utilizing an AI/ML software application and subsequently transmitted to the computing device 100g to be presented by GUI 200a. In further embodiments, GUI 200a can include a list of improvements to or of biomechanics and their associated biomechanic metrics 200b, a first image 200c generated from miss video content (e.g., game video content), and a second image 200d generated from make video content (e.g., practice video content). Further, the list of improvements to biomechanics/biomechanic metrics 200b can include an improvement to biomechanic metric 1 200b-1, an improvement to biomechanic metric 2 200b-2, an improvement to biomechanic metric 3 200b-3, and an overall estimated improvement in performing the athletic skill 200b-4. It should be further understood that other number of images and improvements can be presented, and can be done so based on other categories of events, such as team practice, individual practice, and so forth.

In one or more embodiments, an example for performing an athletic skill can be shooting a basketball from 3-point range. Further, the example biomechanic to be improved can include pre-shot movement (e.g., biomechanic/biomechanic metric 1), jump direction (e.g., biomechanic/biomechanic metric 2), and landing stance (e.g., biomechanic/biomechanic metric 3). Moreover, analyzing the practice video content by the AI/ML software application can determine that player 100b-1 is successful in performing a basketball shot from 3-point range compared to the player's average success rate of the skill (e.g., 50%) when their pre-shot movement is towards the rim (improvement to biomechanic/biomechanic metric 1 200b-1) as opposed to moving left, moving right, moving away from the rim, or stationary; jump direction is slightly forward (improvement to biomechanic/biomechanic metric 2 200b-2) as opposed to straight up, considerably forward, left, right, or backwards; and landing stance to be shoulder width (improvement to biomechanic/biomechanic metric 3) as opposed to wide, staggered left, staggered right, or narrow. For example, for the biomechanic of pre-shot movement, the biomechanic metrics associated with this biomechanic can include the number of successful shots made when pre-shot movement is towards the rim (e.g., biomechanic) divided by the number of shots attempted (e.g., biomechanic metric=success rate). Another example can include the biomechanic of landing stance and the biomechanic metric can include the number of successful shots made when landing stance is shoulder width (e.g., biomechanic) divided by the number of shots attempted (e.g., biomechanic metric=success rate). The particulars or granularity for the biomechanics/biomechanic metrics can be at various levels, including generalized categories such as slightly forward vs. straight up vs. considerably forward, or can be more detailed such as 5-10 degrees forward vs. straight up, vs. greater than 10 degrees forward. Other biomechanic/biomechanic metrics including motion metrics can be tracked, analyzed, and indicated, including degrees of rotation for particular body parts (e.g., release/launch angle is less than 45 degrees as opposed to greater than 45 degrees), distance of body from each other (e.g., elbow positioned within 8 inches of chest, when shot is finished); and so forth. In one embodiments, weightings can be determined for different biomechanic/biomechanic metrics that indicate their influence on overall success rate in performing the athletic skill, such as determining that a wrist rotation of greater than 45 degrees in the follow-through after shooting the basketball has a larger impact on shot success than jumping direction that is forward X degrees. Further, the AI/ML software application can provide on the GUI 200a an overall estimated improvement 200b-4 (16.67%) such that if player 100b-1 is currently shooting 36% from 3-point range, they could possibly improve their shooting percentage to 42% if they implement all the biomechanic/biomechanic metric improvements.

In one or more embodiments, the second image 200d can be generated to be a composite image generated, by the AI/ML software application, from a group of images from the make video content that shows the movements of player 100b-1 when they previously implemented the biomechanic metric improvements (e.g., pre-shot movement is towards the rim, jump direction is slightly forward, and landing stance is shoulder width) in performing the athletic skill (e.g., shooting a basketball from 3-point range) compared to the player's average successful rate of the skill (e.g., 50%). In some embodiments, instead of generating the second image 200d, the AI/ML software application may generate a video clip that can be a composite from a group of images or portions of the make video content that shows the player 100b-1 performing the athletic skill with the biomechanic metric improvements.

In one or more embodiments, the first image 200c can be a composite image generated, by the AI/ML software application, from a group of images from the miss video content that shows the form of player 100b-1 when they are currently implementing the biomechanic metrics (e.g., pre-shot movement, jump direction, and landing stance) in performing the athletic skill (e.g., shooting a basketball from 3-point range)during game play. In some embodiments, instead of generating the first image 200c, the AI/ML software application may generate a video clip that can be a composite from a group of images or portions of the miss video content that shows the player 100b-1 performing the athletic skill with the biomechanic metrics.

In one or more embodiments, the player 100b-2 can view the GUI 200a including first image 200c and second image 200d side-by-side during a break in game play (e.g., halftime, timeout, etc.) to visually understand the way in which they are currently implementing the biomechanic/biomechanic metrics and the way in which they should improve each biomechanic/biomechanic metric accordingly.

Referring to FIG. 2B, in one or more embodiments, system 200-2 comprises GUI 200a that can be displayed or presented on computing device 100g. In some embodiments, the GUI 200a (including any indications and images/video clips) therein can be generated by computing device 100g utilizing an AI/ML software application. In other embodiments, the GUI 200a (including any indications and images/video clips) therein can be generated by server 100h utilizing an AI/ML software application. In further embodiments, GUI 200a can include a list of improvements of biomechanics/biomechanics metrics 200b, a first image 200c generated from miss video content, and a second image 200d generated from make video content. Further, the list of improvements to biomechanics/biomechanic metrics 200b can include a group of biomechanics/biomechanic metrics to improve include an improvement to biomechanic/biomechanic metric 1 200b-1, an improvement to biomechanic/biomechanic metric 2 200b-2, an improvement to biomechanic/biomechanic metric 3 200b-3, and an overall estimated improvement in performing the athletic skill 200b-4.

In one or more embodiments, an example for performing an athletic skill can be shooting a basketball from 3-point range. Further, the example biomechanic/biomechanic metrics to be improved can include pre-shot movement (e.g., biomechanic/biomechanic metric 1), jump direction (e.g., biomechanic/biomechanic metric 2), and landing stance (e.g., biomechanic/biomechanic metric 3). Moreover, analyzing the practice video content by the AI/ML software application can determine that player 100b-1 is successful in performing a basketball shot from 3-point range above a success rate (e.g., 50%) when their pre-shot movement is towards the rim (improvement to biomechanic/biomechanic metric 1 200b-1) as opposed to moving left, moving right, or moving away from the rim; jump direction is slightly forward (improvement to biomechanic/biomechanic metric 2 200b-2) as opposed to straight up, considerably forward, left, or right; and landing stance to be shoulder width (improvement to /biomechanic/biomechanic metric 3) as opposed to wide, staggered left, or staggered right. Further, the AI/ML software application can provide on the GUI 200a an overall estimated improvement 200b-4 (16.67%) such that if player 100b-1 is currently shooting 36% from 3-point range, they could possibly improve their shooting percentage to 42% if they implement all the biomechanic/biomechanic metric improvements.

In one or more embodiments, the second image 200d can be a composite image generated, by the AI/ML software application, from a group of images from the make video content that shows the form of player 100b-1 when they previously implemented the biomechanic/biomechanic metric improvements (e.g., pre-shot movement is towards the rim, jump direction is slightly forward, and landing stance is shoulder width) in performing the athletic skill (e.g., shooting a basketball from 3-point range) above a successful rate (e.g., 50%). In some embodiments, instead of generating the second image 200d, the AI/ML software application may generate a video clip that can be a composite from a group of images or portions of the make video content that shows the player 100b-1 performing the athletic skill with the biomechanic metric improvements.

In one or more embodiments, the first image 200c can be generated from miss video content as a composite image that is generated, by the AI/ML software application, from a group of images that shows the movements of player 100b-1 when they are currently implementing the biomechanic/biomechanic metrics (e.g., pre-shot movement, jump direction, and landing stance) in performing the athletic skill (e.g., shooting a basketball from 3-point range)during game play. In some embodiments, instead of generating the first image 200c, the AI/ML software application may generate a video clip that can be a composite from a group of images or portions of the miss video content that shows the player 100b-1 performing the athletic skill with the biomechanic/biomechanic metrics.

In one or more embodiments, the player 100b-2 can view the GUI 200a including first image 200c and second image 200d in an image overlay 200e during a break in game play (e.g., halftime, timeout, etc.) to visually understand the way in which they are currently implementing the biomechanic metrics and the way in which they should improve each biomechanic metric accordingly.

In one or more embodiments, the player 100b-2 can view the GUI with both the first image and second image to determine the biomechanic metrics they are currently performing during gameplay and the biomechanic metrics they need to perform to improve their performance of the athletic skill based on their own individual strengths discerned by the AI/ML software application analyzing the previous practice content. Moreover, multiple biomechanic metrics can be compiled together to determine which one of the biomechanics has the most significant impact (negative or positive) on the skill. That is, the AI/ML software application creates statistical correlation. In further embodiments, the GUI 200a can present only the biomechanic/biomechanic metric as performed by the player (e.g., from the game video content of practice video content) that can have the largest potential margin for improving their basketball shot. In additional embodiments, the AI/ML software application can generate video content or images of specific drills to improve weaknesses based on objective data from AI/ML analysis.

This allows for the player-100b-2 to make in-game adjustments to immediately improve their performance of the athletic skill instead of waiting for post-game analysis of the game content.

Referring to FIG. 3, in one or more embodiments, system 300 comprises several different components to analyze video content associated with a player performing an athletic skill to determine a group of biomechanics/biomechanic metrics to improve the performance of the athletic skill. Portions and/or the entirety of system 300 can be implemented by computing device 100g or by server 100h. Further, system 300 can comprise an image capture module 300a, an AI/ML software application 300b, a database 300c of AI/ML models, and/or a GUI software application 300d.

In one or more embodiments, the image capture module 300a can be a software application that acquires real-time video content of a player performing an athletic skill from camera sensor 100d-1 and/or camera sensor 100e-1. The AI/ML software application 300b can analyze the video content and determine a group of biomechanic metrics associated with the athletic skill to improve the performance of the athletic skill. In some embodiments, the AI/ML software application 300b can analyze the video content and determine the biomechanic metrics to analyze. In other embodiments, a user (e.g., player, coach, performance expert, etc.) can program or otherwise configure the AI/ML software application 300b with the biomechanics/biomechanic metrics to analyze. Further, the AI/ML software application can generate an image from currently acquired video content to show the player's biomechanics/biomechanic metrics to perform the skill as well as an image generated from previously acquired video content that shows the player's biomechanics/biomechanic metrics.

In one or more embodiments, prior to analyzing the video content, the AI/ML software application 300b can select one or more AI/ML models from the database 300c. In some embodiments, the AI/ML software application 300b can select the one or more AI/ML models based on the athletic skill being analyzed. In other embodiments, the one or more AI/ML models can be selected based on the available processor capacity and/or available memory capacity of the computing device 100g or server 100h implementing the AI/ML software application 300b.

In some embodiments, computing device 100g comprises system 300, and the GUI software application 300d can display the image generated from currently acquired video content as well as an image generated from previously acquired video content. In some embodiments, these images can be displayed simultaneously by the GUI software application side-by-side, as shown in FIG. 2A. In other embodiments, these images can be displayed simultaneously by the GUI software application overlayed on one another, as shown in FIG. 2B. In further embodiments, server 100h comprises system 300, and portions of the GUI software application 300d can span both server 100h and computing device 100g. The portion of GUI software application 300d on server 100h can provide the images to computing device 100g. In addition, the portion of GUI software application 300d on computing device 100g can display the images for the player to view, either side-by-side or overlayed.

Referring to FIG. 4, in one or more embodiments, the AI/ML software application (either on computing device 100g or server 100h) analyzing video content from camera sensor 100d-2 and camera sensor 100e-2 can determine that it cannot adequately analyze the performance of the athletic skill by player 100b-1 because of the position or orientation of camera sensor 100d-2 and camera sensor 100e-2. For example, neither camera sensor does not adequately capture video content of whether player 100b-1 makes or misses their basketball shot. In another example, neither camera sensor does not adequately capture video content of whether player 100b-1 performing the basketball shot or a biomechanic associated with performing the basketball shot. The AI/ML software application can provide instructions to each of camera sensor 100d-2 and camera sensor 100e-2 to adjust its respective position or orientation to have a better perspective in capturing video content of the player 100b-1 performing the athletic skill or recording/determining whether they successfully performed the athletic skill (e.g., successfully making the basketball shot or not). In some embodiments, the instructions to camera sensor 100d-2 can be to move to a higher location. In other embodiments, the instructions to camera sensor 100e-2 can be to adjust orientation to a different angle. Thus, at a different position (e.g., camera sensor 100d-2) and at a different angle/orientation (e.g., camera sensor 100e-2), the subsequently captured/acquired video content of the player performing the athletic skill can be analyzed by the AI/ML software application to determine any biomechanic/biomechanic metrics that can be improved. In one or more embodiments, the instructions and resulting adjustments for the camera sensor 100d-2 and camera sensor 100e-2 can be based on determination a particular biomechanic/biomechanic metric associated with the player that is to be captured (or is not being adequately captured) in order to improve the biomechanic assessment of the player, such as adjusting the orientation and/or position of one of the camera sensors to capture a particular view that shows the angle and/or separation distance between a player's feet when they are on the floor (before jumping), in air (when shot is being released) and back on the floor (when they land after shooting). In one or more embodiments, application of the AI/ML software application to manage or otherwise control the camera sensor 100d-2 and/or camera sensor 100e-2 (e.g., position, orientation, resolution, frame speed, lighting, contrast, and so forth) can provide efficiency in performing the biomechanic assessment of the player, such as ensuring or attempting to ensure that the best possible metrics or data is being captured. The efficiency can also be improved in some embodiments based on remote control of the function, orientation, and positioning of the camera sensor 100d-2 and/or camera sensor 100e-2 that eliminates the need for user interaction with the equipment, which can be a slower process resulting a reduction as to the amount of properly captured data (e.g., from a new camera sensor position or new orientation), such as during a live basketball game.

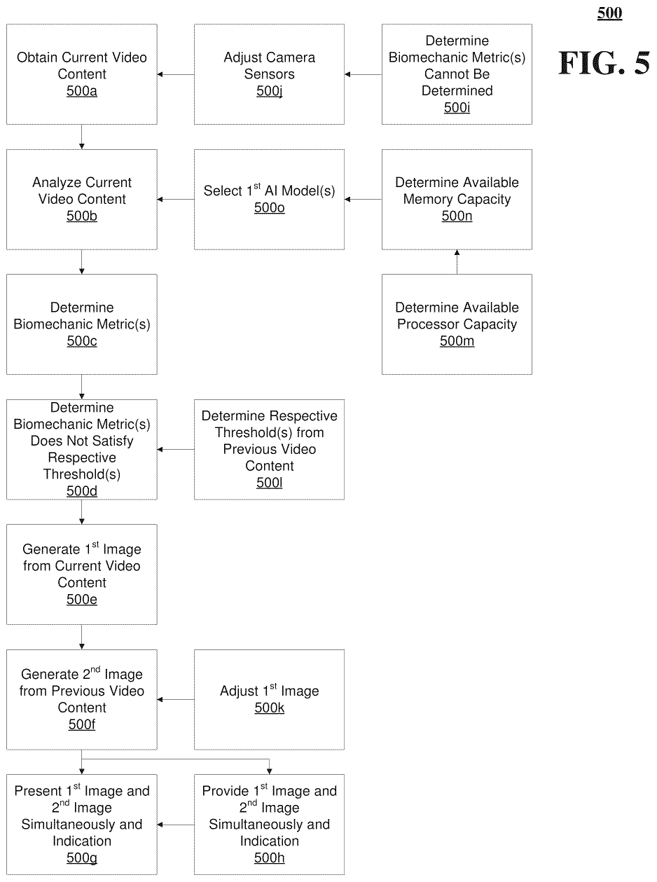

FIG. 5 depicts an illustrative embodiment of a method 500 in accordance with various aspects described herein. Aspects of method 500 can be performed by a computing device and/or server. The method 500 can include the computing device or server, at 500a, obtaining video content of a player repeatedly performing a physical skill during a first time period resulting in current video content. Further, the method 500 can include the computing device or server, at 500b, analyzing the current video content utilizing an AI/ML software application based on previous video content resulting in an analysis. The previous video content comprises other video content of the player repeatedly performing the physical skill during a second time period. The second time period is prior to the first time period. In addition, the method 500 can include the computing device or server, at 500c, determining a group of biomechanic metrics associated with the player performing the physical skill based on the analysis utilizing the AI/ML software application resulting in a first determination. Also, the method 500 can include the computing device or server, at 500d, based on the first determination, determining each biomechanic metric of a portion of the group of biomechanic metrics does not satisfy a respective biomechanic metric success rate from a group of respective biomechanic metric success rates resulting in a second determination.

In one or more embodiments, the method 500 can include the computing device or server, at 500e, based on the second determination, generating a first image of the player performing the physical skill from the current video content. Further, the method 500 can include the computing device or server, at 500f, based on the second determination, generating a second image of the player performing the physical skill from the previous video content. In some embodiments, the method 500 can include the computing device, at 500g, presenting the first image and the second image simultaneously on the device with an indication of the portion of the group of biomechanics that did not satisfy the respective biomechanic metric success rate. In other embodiments, the method 500 can include the server, at 500h, providing the first image and the second image to a computing device over a communication network. The computing device presents the first image and the second image simultaneously on the computing device with an indication of the portion of the group of biomechanics that did not satisfy the respective biomechanic metric success rate. In additional embodiments, the presenting of the first image and the second image simultaneously on the device comprises presenting the first image and the second image side-by-side. In further embodiments, the presenting of the first image and the second image simultaneously on the device comprises presenting the second image overlaid onto the first image. In some embodiments, instead of a first image generated from current video content or a second image generated from previous video content, a first video clip can be generated from current video content and a second video clip can generated from previous video content. Further, both the first video clip and the second video clip can be provided to the GUI application on the computing device to be presented to a player simultaneously, side-by-side or overlayed.

In one or more embodiments, the method 500 can include the computing device or server, at 500k, adjusting the first image based on the portion of the group of biomechanics. In some embodiments, the generating the second image comprises adjusting the first image based on the portion of the group of biomechanics. In some embodiments, generating of the second video clip can comprise adjusting the first video clip based on the portion of the group of biomechanics.

In one or more embodiments, the recording of the video content of the player repeatedly performing the physical skill during the first time period can be done with a group of camera sensors. Each of the group of camera sensors are at a respective first position. Further, the method 500 can include the computing device or server, at 500i, determining that the group of biomechanic metrics cannot be determined from a portion of the current video content utilizing the AI/ML software application resulting in a third determination. In addition, the method 500 can include the computing device or server, at 500j, based on the third determination, adjusting a portion of the group of camera sensors from the respective first position to a respective second position. The adjusting a portion of the group of camera sensors can include providing instructions to each of the group of camera sensors that indicate to change their respective position or orientation (any 360-degree adjustment of position, up and down, laterally, or back and forth).

In one or more embodiments, the method 500 can include the computing device or server, at 500l, determining the group of respective biomechanic metric success rates from the previous video content utilizing the AI/ML software application. For example, in shooting a basketball from 3-point range, one biomechanic can be pre-shot movement. Further, the biomechanic metrics for this biomechanic can include left, right, towards the rim, away from the rim, or stationary. Moreover, the AI/ML software application can determine that the player is most successful in making a basketball shot from 3-point range when they conduct pre-shot movement towards the rim. That is, the player makes a shot from 3-point range 78% of the time when their pre-shot movement is towards the rim. Thus, the biomechanic metric success rate for pre-shot movement towards the rim is 78%. In another example, another biomechanic can be jump direction. The biomechanic metric for this biomechanic can include straight up, slightly forward, considerably more forward, left, right, and backwards. Moreover, the AI/ML software application can determine that the player is most successful in make a basketball shot from 3-point range when they conduct a jump straight up. That is, the player makes a shot from 3-point range 74.1% (e.g., biomechanic metric success rate) of the time when they jump straight up. In a further example, a further biomechanic can be landing stance. The biomechanic metric for this biomechanic can include shoulder width, wide, staggered left, staggered right, and narrow. Moreover, the AI/ML software application can determine that the player is most successful in making a basketball shot from 3-point range when they set their landing stance shoulder width. That is, the player makes a shot from 3-point range 77.1% (e.g., biomechanic metric success rate) of the time when they set their landing stance shoulder width.

In one or more embodiments, the method 500 can include the computing device or server, at 500m, determining available processing capacity of the processing system resulting in a fourth determination. In addition, the method 500 can include the computing device or server, at 500n, determining available memory capacity of the memory resulting in a fifth determination. Also, the method 500 can include the computing device or server, at 500o, based on the fourth determination and the fifth determination, selecting the first group of AI/ML models from a second group of AI/ML models.

In one or more embodiments, the physical skill comprises a basketball shot and the group of biomechanic/biomechanic metrics can include shot type, shot preparation, pre-shot movement, footwork, toes pointed, stance, group of release time, reception of pass, pass details, landing knees, eye gaze, jump direction, landing stance, landing feet, landing movement, follow through, follow through details, wrist, guide hand, discipline, result, and swish.

In one or more embodiments, the physical skill or athletic skill can comprise not only a basketball shot, but also, but not limited to, passing a basketball, defending a basketball player, blocking a basketball shot, shooting a soccer ball, passing a soccer ball defending a soccer player, saving a soccer ball by goalkeeper, throwing a football, catching a football, blocking in football, tacking in football, kicking a football, punting a football, swing a bat in baseball, catching a baseball, pitching in baseball, sliding in baseball, skating in hocket, shooting in hockey, passing in hockey, checking in hockey, defending in hockey, saving a puck by a goalkeeper in hockey, swing a tennis racquet, swinging a golf club, or performing any other physical skill or athletic skill.

While for purposes of simplicity of explanation, the respective processes are shown and described as a series of blocks in FIG. 5, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methods described herein. In some embodiments, one or more blocks can be performed in response to one or more other blocks.

Portions of some embodiments can be combined with portions of other embodiments.

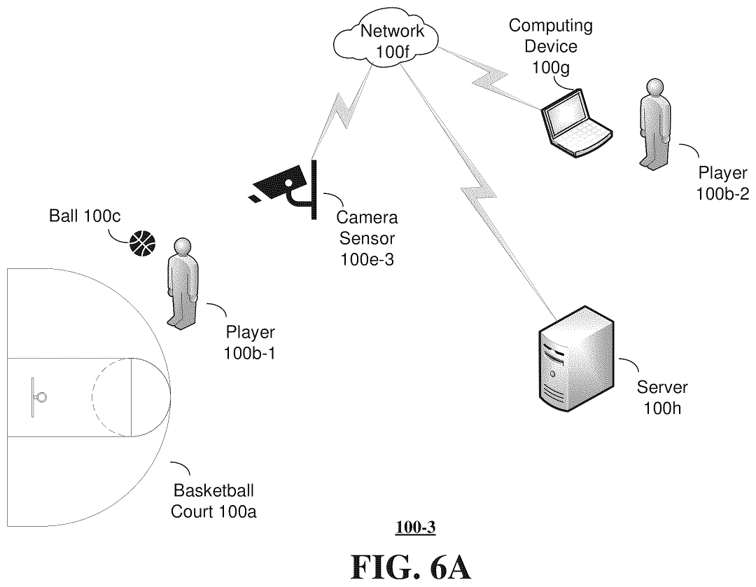

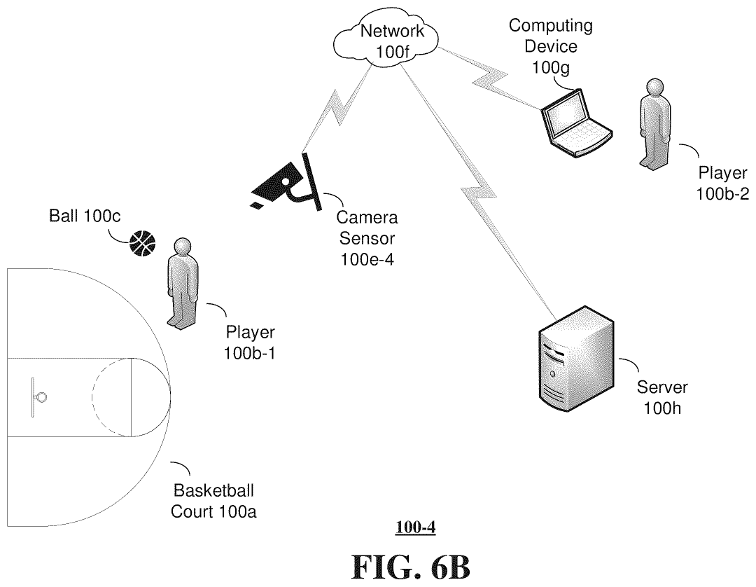

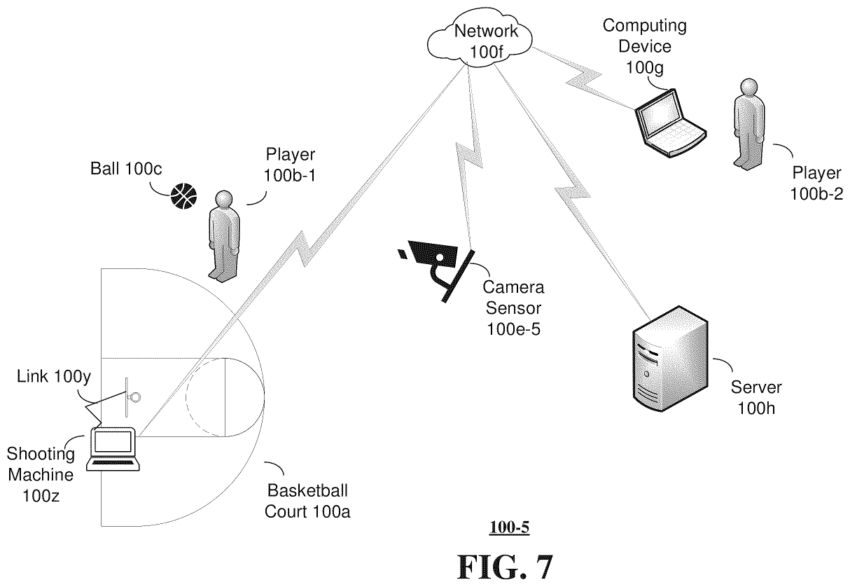

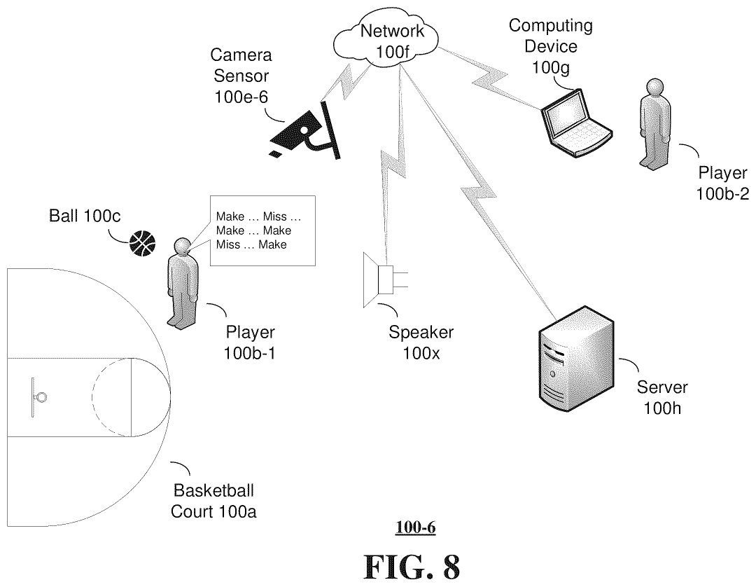

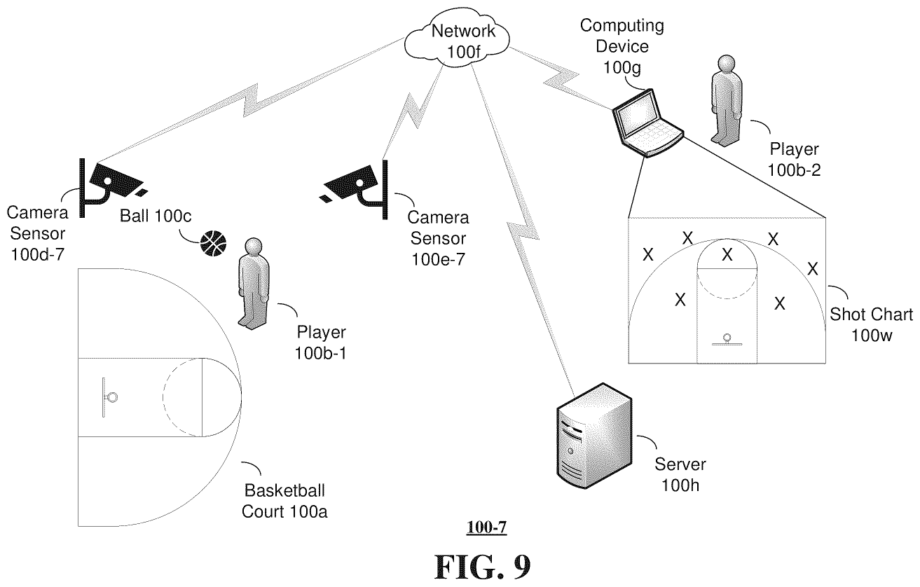

Referring to FIG. 6A, in one or more embodiments, system 100 includes a basketball court 100a, player 100b-1 shooting a basketball 100c, a camera sensor 100e-3 positioned/oriented to recording image content or video content of player 100b-1 shooting basketball 100c. Further, camera sensor 100e-3 is communicatively coupled to computing device 100g, and server 100h over communication network 100f. Thus, system 100-3 can include all the components of system 100-1 but instead of system 100-1 including two cameras, namely camera sensor 100d-1 and camera sensor 100e-1, system 100-3 includes only one camera, namely camera sensor 100e-3.

In one or more embodiments, camera sensor 100e-3 is positioned/oriented such that it records player 100b-1 shooting the ball 100c at the basket and records whether player 100b-1 successfully shoots the ball 100c or does not successfully shoot the ball (e.g., to later determine the success rate). In further embodiments, the computing device 100g can obtain video content of a player 100b-1 repeatedly performing shooting a basketball 100c during gameplay resulting in current video content utilizing camera sensor 100e-3. The camera sensor 100e-3 is oriented in a first position. In some embodiments, prior to analyzing the current video content, an AI software application implemented on either computing device 100g or server 100h can determine that a group of biomechanics/biomechanic metrics cannot be determined from a portion of the current video content resulting in a determination. Further, based on the determination, the computing device 100g or the server 100h can provide instructions to the camera sensor 100e-3 to adjust the camera sensor 100e-3 from the first position to a second position. That is, upon analyzing the current video content, the AI software application determines that it cannot record the entire body of the players 100b-1 while taking a shot and record whether the player 100b-1 successfully made the shot. Based on the determination, the computing device 100g can adjust or provide instructions to the camera sensor to adjust from the first position to a second position, as shown in FIG. 6B.

In one or more embodiments, upon analyzing the current video content, the AI software application can determine that it can record the entire body of the players 100b-1 while taking a shot and record whether the player 100b-1 successfully made the shot. Further, computing device 100g can analyze the current video content utilizing the AI software application based on previous video content resulting in an analysis. The previous video content comprises other video content of the player repeatedly making a shot during a second time period. The second time period is prior to the first time period. In addition, the computing device 100g can determine a group of biomechanic metrics associated with the player performing the basketball shot based on the analysis utilizing the AI software application resulting in a determination. Also, based on the determination, determining each biomechanic metric of a portion of the group of biomechanic metrics does not satisfy a respective biomechanic metric success rate from a group of respective biomechanic metric success rates resulting in another determination. Based on this other determination, generating a first image of the player 100b-1 shooting the basketball 100c from the current video content. Further, based on this other determination, generating a second image of the player 100b-1 shooting the basketball 100c from the previous video content. In addition, the computing device 100g can present the first image and the second image simultaneously on the computing device 100g with an indication of the portion of the group of biomechanics that did not satisfy the respective biomechanic metric success rate.

In one or more embodiments, the generating the second image by computing device 100g comprises adjusting the first image based on the portion of the group of biomechanics by the computing device 100g. In addition, the presenting of the first image and the second image simultaneously on the computing device 100g comprises presenting the first image and the second image side-by-side. Also, the presenting of the first image and the second image simultaneously on computing device 100g can comprise presenting the second image overlaid onto the first image. In some embodiments, computing device 100g can determine the group of respective biomechanic metric success rates from the previous video content utilizing the AI software application.

In one or more embodiments, camera 100e-3 can be focused on, and capturing images of, player 100b-1 while they are shooting basketball 100c and camera 100e-3 may not be focused on, and not capturing images of the rim to determine whether the basketball shot attempted by player 100b-1 has been made or missed. Instead, in some embodiments, a sensor (e.g., motion sensor, movement sensor, image sensor, etc.) can be coupled to, in communication with, or otherwise associated with the rim to determine whether the attempted basketball shot has been made or missed. In further embodiments, the sensor can be communicatively coupled with computing device 100g and/or server 100h to provide data indicating whether a group of attempted basketball shots are made or missed. In addition, camera sensor 100e-3 can provide captured images of player 100b-1 shooting the basketball for each attempted basketball shot Further, the AI/ML software application on either computing device 100g and/or server 100h can analyze the captured images as well as the sensor data to determine the number/percentage of makes or misses associated with the group of basketball shots as well as identify the images that associated with the made basketball shots and the images associated with the missed basketball shots. These images can be displayed to the player 100b-2 so as they can review the biomechanics associated with the missed basketball shots as well as the biomechanics associated with made basketball shots and improve accordingly.

Referring to FIG. 7, in one or more embodiments, system 100-5 includes a basketball court 100a, player 100b-1 shooting a basketball 100c, a camera sensor 100e-5 positioned/oriented to recording image content or video content of player 100b-1 shooting basketball 100c. Further, camera sensor 100e-5 and shooting machine 100z are communicatively coupled to computing device 100g, and server 100h over communication network 100f. In addition, the shooting machine 100z can be communicatively to the basketball hoop on basketball court 100c via communication link 100y. Thus, system 100-3 can include all the components of system 100-1 but instead of system 100-1 including two cameras, namely camera sensor 100d-1 and camera sensor 100e-1, system 100-3 includes only one camera, namely camera sensor 100e-3. Moreover, the system 100-5 can include shooting machine 100z.

In one or more embodiments, system 100-5 that utilizes an AI software application on either computing device 100g or server 100h to analyze video content received from camera sensor of player 100b-1 shooting basketball 100c to determine the player's success rate and provide any information to improve the success rate. However, while system 100-1 includes two camera sensors, one camera sensor recording the player shooting the basketball and another camera sensor recording whether the basketball shot was a success or not (e.g., can be used to determine the success rate), system 100-5 includes one camera sensor 100e-5 recording the player 100b-1 shooting the basketball and the shooting machines 100z determining whether the basketball shot was a success or not utilizing one or more sensors in proximity to the basketball hoop (e.g., sensor data can be used to determine success rate).

In one or more embodiments, computing device 100g can obtain video content of player 100b-1 repeatedly performing a physical skill (e.g., shooting a basketball) during a first time period resulting in current video content utilizing camera sensor 100e-5. The camera sensor is oriented in a first position. Further, computing device 100g can obtain sensor data from a physical skill practice machine (e.g., shooting machine 100z). The sensor data indicates correctly performing and incorrectly performing the physical skill repeatedly (e.g., success rate of shooting the basketball 100c). In addition, the computing device 100g can analyze the current video content and the sensor data utilizing an AI software application based on previous video content resulting in an analysis. The previous video content comprises other video content of the player repeatedly performing the physical skill (e.g., shooting the basketball 100c) during a second time period. The second time period is prior to the first time period. Also, the computing device 100g can determine a group of biomechanic metrics associated with the player performing the physical skill (e.g., shooting the basketball 100c) based on the analysis utilizing the AI software application resulting in a first determination.

In one or more embodiments, based on the first determination, computing device 100g can determine each biomechanic metric of a portion of the group of biomechanic metrics does not satisfy a respective biomechanic metric success rate from a group of respective biomechanic metric success rates resulting in a second determination. Further, based on the second determination, computing device 100g can generate a first image of the player performing the physical skill (e.g., the shooting of basketball 100c) from the current video content. In addition, based on the second determination, computing device 100g can generate a second image of the player performing the physical skill (e.g., shooting basketball 100c) from the previous video content. Also, the computing device 100g can present the first image and the second image simultaneously on the device with an indication of the portion of the group of biomechanics that did not satisfy the respective biomechanic metric success rate.

In one or more embodiments, the generating, by the computing device 100g, the second image comprises adjusting, by the computing device 100g, the first image based on the portion of the group of biomechanics. In other embodiments, the presenting of the first image and the second image simultaneously on the computing device 100g comprises presenting the first image and the second image side-by-side. In further embodiments, the presenting of the first image and the second image simultaneously on the device comprises presenting the second image overlaid onto the first image. Further, the computing device 100g, can determine the group of respective biomechanic metric success rates from the previous video content utilizing the AI software application.

In one or more embodiments, the server 100h can provide the same functions as computing device 100g described herein and provide the first image and second image to the computing device 100g over network 100f such that computing device 100g can present the first image and the second image simultaneously, side-by-side or overlaid.

Referring to FIG. 8, in one or more embodiments, system 100-6 includes a basketball court 100a, player 100b-1 shooting a basketball 100c, a camera sensor 100e-6 positioned/oriented to recording image content or video content of player 100b-1 shooting basketball 100c. Further, camera sensor 100e-3 is communicatively coupled to computing device 100g, and server 100h over communication network 100f. Thus, system 100-3 can include all the components of system 100-1 but instead of system 100-1 including two cameras, namely camera sensor 100d-1 and camera sensor 100e-1, system 100-3 includes only one camera, namely camera sensor 100e-3. In addition, system 100-6 includes a speaker to record audio content spoken by player 100b-1.

In one or more embodiments, system 100-6 utilizes an AI software application on either computing device 100g or server 100h to analyze video content received from camera sensor of player 100b-1 shooting basketball 100c to determine the player's success rate and provide any information to improve the success rate. However, while system 100-1 includes two camera sensors, one camera sensor recording the player shooting the basketball and another camera sensor recording whether the basketball shot was a success or not (e.g., can be used to determine the success rate), system 100-5 includes one camera sensor 100e-5 recording the basketball shot performed by the player 100b-1 and player 100b-1 speaking on whether each basketball shot is a success or not. In some embodiments, player 100b-1 can say “make” to indicate that a basketball shot was a success and player 100b-1 can say “miss” to indicate that a basketball shot was not a success. Further, such audio content spoken by player 100b-1 can be recorded by speaker 100x and provide to computing device 100g to be analyzed by the AI software application.

In one or more embodiments, the computing device 100g can obtain video content of player 100b-1 repeatedly performing a physical skill (e.g., shooting a basketball) during a first time period resulting in current video content utilizing camera sensor 100e-6. Further, camera sensor 100e-6 is oriented in a first position. In addition, computing device 100g can obtain voice input from player 100b-1, via speaker 100x, utilizing voice recognition techniques or technology. The voice input indicates correctly performing and incorrectly performing the physical skill (shooting basketball) repeatedly. Also, computing device 100g can analyze the current video content and the voice input utilizing an AI software application based on previous video content resulting in an analysis. The previous video content comprises other video content of the player repeatedly performing the physical skill (e.g., shooting basketball) during a second time period. The second time period is prior to the first time period.

In one or more embodiments, computing device 100g can determine a group of biomechanic metrics associated with the player performing the physical skill (e.g., shooting basketball) based on the analysis utilizing the AI software application resulting in a first determination. Further, based on the first determination, computing device 100g can determine each biomechanic metric of a portion of the group of biomechanic metrics does not satisfy a respective biomechanic metric success rate from a group of respective biomechanic metric success rates resulting in a second determination. In addition, based on the second determination, computing device 100g can generate a first image of player 100b-1 performing the physical skill (e.g., shooting a basketball) from the current video content. In addition, based on the second determination, computing device 100g can generate a second image of player 100b-1 performing the physical skill (e.g., shooting the basketball) from the previous video content. Also, the computing device 100g can present the first image and the second image simultaneously on computing device 100g to player 100b-2 with an indication of the portion of the group of biomechanics that did not satisfy the respective biomechanic metric success rate.

In one or more embodiments, the generating of the second image comprises adjusting the first image based on the portion of the group of biomechanics. Further, the presenting of the first image and the second image simultaneously on the computing device 100g comprises presenting the first image and the second image side-by-side. In addition, the presenting of the first image and the second image simultaneously on the computing device 100g comprises presenting the second image overlaid onto the first image. Also, computing device 100g can determine the group of respective biomechanic metric success rates from the previous video content utilizing the AI software application.

In one or more embodiments, the server 100h can provide the same functions as computing device 100g described herein and provide the first image and second image to the computing device 100g over network 100f such that computing device 100g can present the first image and the second image simultaneously, side-by-side or overlaid.

In one or more embodiments, instead of speaking “make” or “miss” to indicate whether player 100b-1 has made an attempted basketball shot or missed an attempted basketball shot, player 100b-1 can make a first hand signal (e.g., thumbs up) or can make a second hand signal (e.g., thumbs down). The image of the first hand signal or the second hand signal can be captured by camera sensor 100d-6 and the images associated with the first hand signals and the second hand signals can be provided to computing device 100g and/or server 100h. Further, the AI/ML software application can analyze the biomechanics associated with a portion of the basketball shots and associated the biomechanics with the first hand signal and determine that the associated basketball shots are made basketball shots. In addition, the AI/ML software application can analyze the biomechanics associated with another portion of the basketball shots and associated the biomechanics with the second hand signal and determine that the associated basketball shots are missed basketball shots, and can provide images of their biomechanics accordingly via computing device 100g, thereby providing recommendations for improvement to player 100b-2.

In one or more embodiments, instead of speaking “make” or “miss” to indicate whether player 100b-1 has made a basketball shot or missed a basketball shot, player 100b-1 can make a first clapping sound (e.g., two claps) or can make a second clapping sound (e.g., one clap). The audio of the first clapping sound or the second clapping sound can be captured by speaker 100x and the associated audio content can be provided to computing device 100g and/or server 100h, thereby generating a group of audio content. Further, the AI/ML software application can analyze the biomechanics associated with the shooting the basketball and with the audio content of the first clapping signal and determine that the associated basketball shots are made basketball shots. In addition, the AI/ML software application can analyze the biomechanics associated with the shooting the basketball and associated audio content of the second hand signal and determine that the associated basketball shots are missed basketball shots, and can provide images of their biomechanics accordingly via computing device 100g to improve the basketball shot of player 100b-2 accordingly.

In one or more embodiments, player 100b-1 can have a mobile device (e.g., a smartwatch, mobile phone, tablet computer, etc.) that includes a mobile application associated with the AI/ML software application on the computing device 100g and/or server 100h. The mobile device can provide a tactile interface (e.g., a touchscreen, switch, button, dial, etc.) such that player 100b-1 can provide first user-generated input (e.g., touching a first icon on the mobile application) to indicate a made basketball shot and can provide a second user-generated input (e.g., touching a second icon on the mobile application) indicate a missed basketball shot. The group of user-generated input associated with the group of basketball shots that indicate made basketball shots and missed basketball shots can be provided to the AI/ML software application on either computing device 100g and/or server 100h. Further, the AI/ML software application can analyze the images provided by camera sensor 100e-6 to either computing device 100g or server 100h for biomechanics associated with the shooting the basketball with the group of user-generated input (or a portion thereof associated with the first user-generated input) to determine that the associated basketball shots are made basketball shots. In addition, the AI/ML software application can analyze the images provided by camera sensor 100e-6 to either computing device 100g or server 100h for the biomechanics associated with the shooting the basketball with the group of user-generated input (or a portion thereof associated with the second user-generated input) to determine that the associated basketball shots are missed basketball shots, and can provide images of their biomechanics accordingly via computing device 100g to improve the basketball shot of player 100b-2.

Referring to FIG. 9, in one or more embodiments, system 100-7 includes a basketball court 100a, player 100b-1 shooting a basketball 100c, a camera sensor 100d-7 positioned/oriented to recording image content or video content of player 100b-1 shooting basketball 100c and a camera sensor 100e-7 positioned/oriented to recording image content or video content that the basketball shot was a success or not. Further, camera sensor 100d-7 and camera sensor 100e-7 are communicatively coupled to computing device 100g, and server 100h over communication network 100f.

In one or more embodiments, system 100-6 utilizes an AI software application on either computing device 100g or server 100h to analyze video content received from camera sensor of player 100b-1 shooting basketball 100c to determine the player's success rate and provide any information to improve the success rate. However, AI software application executing on computing device 100g can provide a gamification practice session. That is, the AI software application can provide a graphical user interface (GUI) on computing device 100g multiple simulated gameplay scenarios. In some embodiments, each simulated gameplay scenario can be depicted by a shot chart 100w. In further embodiments, the multiple simulated gameplay scenarios can be generated by analyzing stored video content of a player practicing shooting a basketball and determining that each simulated gameplay scenario can improve a biomechanic metric associated with shooting the basketball. In addition, the player 100b-2 can select, via user-generated input, one of the multiple simulated gameplay scenarios and display an associated shot chart 100w, accordingly. In some embodiments, the shot chart 100w can comprise an animated shot chart that animates through each shot in the shot chart 100w. In other embodiments, the animated shot chart presents an indication of each of the group of basketball shots via animation

In one or more embodiments, player 100b-1 can perform a practice session for shooting a basketball according to the shot chart 100w and camera sensor 100d-7 as well as camera sensor 100e-7 can record the practice session accordingly. Further, the recorded content can be provided to computing device 100g for analysis by the AI software application to determine any improvements for shooting the basketball by player 100b-1 and provide suggest the improvements accordingly.

In one or more embodiments, computing device 100g can provide GUI on computing device 100g that presents a group of simulated gameplay scenarios to player 100b-2. Further, the computing device 100g can receive user-generated input from the GUI as provided player 100b-2. The user-generated input indicates a first simulated gameplay scenario from the group of simulated gameplay scenarios. In addition, the computing device 100g can present the first simulated gameplay scenario on the GUI to the player 100b-2.

In one or more embodiments, computing device 100g can obtain current video content of the player repeatedly performing a physical skill (e.g., shooting basketball) during a first time period according to the first simulated gameplay scenario resulting in current video content. A portion of the video content can be from camera sensor 100d-7 and another portion of the video content can be from camera sensor 100e-7. Further, the computing device 100g can analyze the current video content utilizing an AI software application based on previous video content resulting in an analysis. The previous video content comprises other video content of the player repeatedly performing the physical skill (e.g., shooting a basketball) during a second time period. The second time period is prior to the first time period. In addition, the computing device 100g can determine a group of biomechanic metrics associated with the player performing the physical skill based on the analysis utilizing the AI software application resulting in a first determination. Also, based on the first determination, the computing device 100g can determine each biomechanic metric of a portion of the group of biomechanic metrics does not satisfy a respective biomechanic metric success rate from a group of respective biomechanic metric success rates resulting in a second determination. Further, based on the second determination, the computing device 100g can determine a second simulated gameplay scenario from the group of simulated gameplay scenarios utilizing the AI software application. The second simulated gameplay scenario improves a first biomechanic metric of the portion of the group of biomechanic metrics. In addition, the computing device 100g can present the second simulated gameplay scenario on the GUI to the player.

In one or more embodiments, based on the second determination, computing device 100g can determine a first biomechanic associated with the first biomechanic metric resulting in a third determination. Further, based on the third determination, the computing device 100g can identify a portion of the previous video content associated with the first biomechanic. In addition, the computing device 100g can present the portion of the previous video content on the GUI to the player to indicate how to improve the success rate of performing the physical skill (e.g., shooting the basketball).

In one or more embodiments, the presenting of the portion of the previous video content comprises presenting the portion of the previous video content on the GUI simultaneously with presenting the second simulated gameplay scenario on the GUI. Further, the computing device 100g can determine the group of respective biomechanic metric success rates from the previous video content utilizing the AI software application.

In one or more embodiments, the server 100h can provide the same functions as computing device 100g described herein. For example, the server 100h can generate the multiple simulated gameplay scenarios and provide them to the computing device 100g to be presented on a GUI on computing device 100g to player 100b-2.

In one or more embodiments, the AI software application on either computing device 100g or server 100h can provide mobile application on a mobile device (e.g., smart watch, mobile phone, tablet computer, etc.) with a shot chart 100w. In further embodiments, the shot chart 100w can be part of a gamification embodiment of the AI software application. The gamification embodiment can include a computer-generated opponent that makes or misses the same or similar shots as the shot chart 100w. The computer-generated opponent can simulate a real professional, college, or other player. Further, the gamification embodiments can include an increasing level of difficulty of shot within the shot chart 100w as the basketball player 100b-1 progress along the shot chart 100w. In some embodiments, the AI software application can keep track of a won/loss record of player 100b-1 for each game played against such a computer-generated opponent. In further embodiments, the AI software application can keep track the won/loss record of games of player 100b-1 played against a computer-generated opponent or a group of computer-generated opponents that can correspond to a simulated season of games. The AI software application can receive captured data (e.g., image data, audio content, user-generated input, etc.), as described herein, of the makes and misses of the basketball shots attempted by the player 100b-1 according to the shot chart 100w. Further, the AI software application can provide improvements and adjustments of the biomechanics of player 100b-1 as described herein accordingly.

Note, embodiments that describe an AI software application can also utilize an ML software application to provide the same or similar functions as the AI software application. Further, embodiments that describe an ML software application can also utilize an AI software application to provide the same or similar functions as the ML software application.

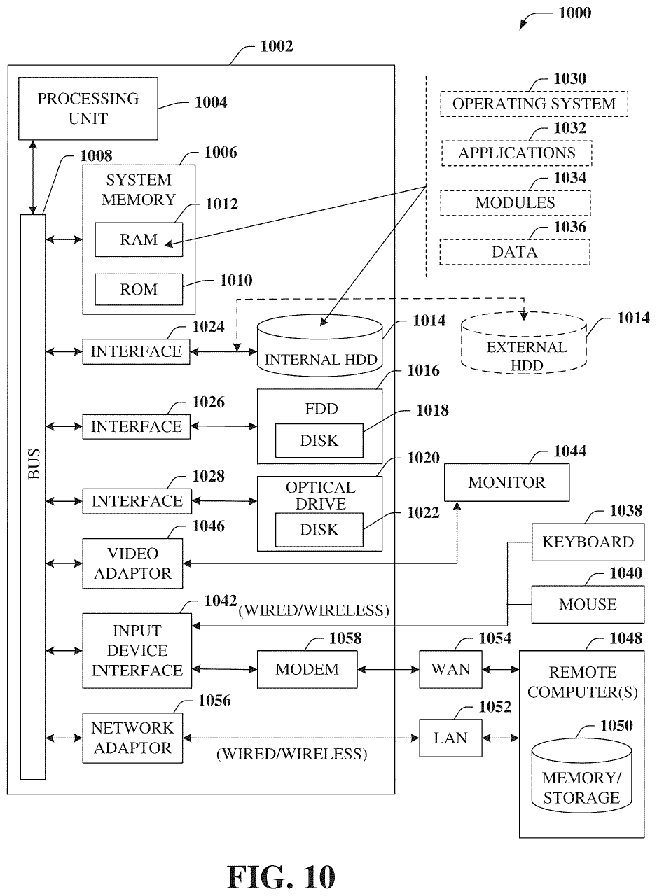

Turning now to FIG. 10, there is illustrated a block diagram of a computing environment in accordance with various aspects described herein. In order to provide additional context for various embodiments of the embodiments described herein, FIG. 10 and the following discussion are intended to provide a brief, general description of a suitable computing environment 1000 in which the various embodiments of the subject disclosure can be implemented. For example, computing environment 1000 can facilitate in whole or in part capturing video content of a player repeatedly performing an athletic skill to be analyzed to determine biomechanic adjustments to improve performance of the athletic skill. Each of camera sensor 100d-1, camera sensor 100e-1, camera sensor 100d-2, camera sensor 100e-2, camera sensor 100e-3, camera sensor 100e-4, camera sensor 100e-5, shooting machine 100z, speaker 100x, computing device 100g, and server 100h can comprise aspects of computing environment 1000.

Generally, program modules comprise routines, programs, components, data structures, etc., that perform particular tasks or implement particular abstract data types. Moreover, those skilled in the art will appreciate that the methods can be practiced with other computer system configurations, comprising single-processor or multiprocessor computer systems, minicomputers, mainframe computers, as well as personal computers, hand-held computing devices (including a phone), microprocessor-based or programmable consumer electronics, and the like, each of which can be operatively coupled to one or more associated devices.

As used herein, a processing circuit includes one or more processors as well as other application specific circuits such as an application specific integrated circuit, digital logic circuit, state machine, programmable gate array or other circuit that processes input signals or data and that produces output signals or data in response thereto. It should be noted that while any functions and features described herein in association with the operation of a processor could likewise be performed by a processing circuit.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically comprise a variety of media, which can comprise computer-readable storage media and/or communications media, which two terms are used herein differently from one another as follows. Computer-readable storage media can be any available storage media that can be accessed by the computer and comprises both volatile and nonvolatile media, removable and non-removable media. By way of example, and not limitation, computer-readable storage media can be implemented in connection with any method or technology for storage of information such as computer-readable instructions, program modules, structured data or unstructured data.

Computer-readable storage media can comprise, but are not limited to, random access memory (RAM), read only memory (ROM), electrically erasable programmable read only memory (EEPROM), flash memory or other memory technology, compact disk read only memory (CD ROM), digital versatile disk (DVD) or other optical disk storage, magnetic cassettes, magnetic tape, magnetic disk storage or other magnetic storage devices or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and comprises any information delivery or transport media. The term “modulated data signal” or signals refers to a signal that has one or more of its characteristics set or changed in such a manner as to encode information in one or more signals. By way of example, and not limitation, communication media comprise wired media, such as a wired network or direct-wired connection, and wireless media such as acoustic, RF, infrared and other wireless media.

With reference again to FIG. 10, the example environment can comprise a computer 1002, the computer 1002 comprising a processing unit 1004, a system memory 1006 and a system bus 1008. The system bus 1008 couples system components including, but not limited to, the system memory 1006 to the processing unit 1004. The processing unit 1004 can be any of various commercially available processors. Dual microprocessors and other multiprocessor architectures can also be employed as the processing unit 1004.

The system bus 1008 can be any of several types of bus structure that can further interconnect to a memory bus (with or without a memory controller), a peripheral bus, and a local bus using any of a variety of commercially available bus architectures. The system memory 1006 comprises ROM 1010 and RAM 1012. A basic input/output system (BIOS) can be stored in a non-volatile memory such as ROM, erasable programmable read only memory (EPROM), EEPROM, which BIOS contains the basic routines that help to transfer information between elements within the computer 1002, such as during startup. The RAM 1012 can also comprise a high-speed RAM such as static RAM for caching data.

The computer 1002 further comprises an internal hard disk drive (HDD) 1014 (e.g., EIDE, SATA), which internal HDD 614 can also be configured for external use in a suitable chassis (not shown), a magnetic floppy disk drive (FDD) 1016, (e.g., to read from or write to a removable diskette 1018) and an optical disk drive 1020, (e.g., reading a CD-ROM disk 1022 or, to read from or write to other high-capacity optical media such as the DVD). The HDD 1014, magnetic FDD 1016 and optical disk drive 1020 can be connected to the system bus 1008 by a hard disk drive interface 1024, a magnetic disk drive interface 1026 and an optical drive interface 1028, respectively. The hard disk drive interface 1024 for external drive implementations comprises at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1002, the drives and storage media accommodate the storage of any data in a suitable digital format. Although the description of computer-readable storage media above refers to a hard disk drive (HDD), a removable magnetic diskette, and a removable optical media such as a CD or DVD, it should be appreciated by those skilled in the art that other types of storage media which are readable by a computer, such as zip drives, magnetic cassettes, flash memory cards, cartridges, and the like, can also be used in the example operating environment, and further, that any such storage media can contain computer-executable instructions for performing the methods described herein.

A number of program modules can be stored in the drives and RAM 1012, comprising an operating system 1030, one or more application programs 1032, other program modules 1034 and program data 1036. All or portions of the operating system, applications, modules, and/or data can also be cached in the RAM 1012. The systems and methods described herein can be implemented utilizing various commercially available operating systems or combinations of operating systems.

A user can enter commands and information into the computer 1002 through one or more wired/wireless input devices, e.g., a keyboard 1038 and a pointing device, such as a mouse 1040. Other input devices (not shown) can comprise a microphone, an infrared (IR) remote control, a joystick, a game pad, a stylus pen, touch screen or the like. These and other input devices are often connected to the processing unit 1004 through an input device interface 1042 that can be coupled to the system bus 1008, but can be connected by other interfaces, such as a parallel port, an IEEE 1394 serial port, a game port, a universal serial bus (USB) port, an IR interface, etc.

A monitor 1044 or other type of display device can be also connected to the system bus 1008 via an interface, such as a video adapter 1046. It will also be appreciated that in alternative embodiments, a monitor 1044 can also be any display device (e.g., another computer having a display, a smart phone, a tablet computer, etc.) for receiving display information associated with computer 1002 via any communication means, including via the Internet and cloud-based networks. In addition to the monitor 1044, a computer typically comprises other peripheral output devices (not shown), such as speakers, printers, etc.

The computer 1002 can operate in a networked environment using logical connections via wired and/or wireless communications to one or more remote computers, such as a remote computer(s) 1048. The remote computer(s) 1048 can be a workstation, a server computer, a router, a personal computer, portable computer, microprocessor-based entertainment appliance, a peer device or other common network node, and typically comprises many or all of the elements described relative to the computer 1002, although, for purposes of brevity, only a remote memory/storage device 1050 is illustrated. The logical connections depicted comprise wired/wireless connectivity to a local area network (LAN) 1052 and/or larger networks, e.g., a wide area network (WAN) 1054. Such LAN and WAN networking environments are commonplace in offices and companies, and facilitate enterprise-wide computer networks, such as intranets, all of which can connect to a global communications network, e.g., the Internet.

When used in a LAN networking environment, the computer 1002 can be connected to the LAN 1052 through a wired and/or wireless communication network interface or adapter 1056. The adapter 1056 can facilitate wired or wireless communication to the LAN 1052, which can also comprise a wireless AP disposed thereon for communicating with the adapter 1056.

When used in a WAN networking environment, the computer 1002 can comprise a modem 1058 or can be connected to a communications server on the WAN 1054 or has other means for establishing communications over the WAN 1054, such as by way of the Internet. The modem 1058, which can be internal or external and a wired or wireless device, can be connected to the system bus 1008 via the input device interface 1042. In a networked environment, program modules depicted relative to the computer 1002 or portions thereof, can be stored in the remote memory/storage device 1050. It will be appreciated that the network connections shown are example and other means of establishing a communications link between the computers can be used.

The computer 1002 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This can comprise Wireless Fidelity (Wi-Fi) and BLUETOOTH® wireless technologies. Thus, the communication can be a predefined structure as with a conventional network or simply an ad hoc communication between at least two devices.

Wi-Fi can allow connection to the Internet from a couch at home, a bed in a hotel room or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE 802.11 (a, b, g, n, ac, ag, etc.) to provide secure, reliable, fast wireless connectivity. A Wi-Fi network can be used to connect computers to each other, to the Internet, and to wired networks (which can use IEEE 802.3 or Ethernet). Wi-Fi networks operate in the unlicensed 2.4 and 5 GHz radio bands for example or with products that contain both bands (dual band), so the networks can provide real-world performance similar to the basic 10BaseT wired Ethernet networks used in many offices.

What has been described above includes mere examples of various embodiments. It is, of course, not possible to describe every conceivable combination of components or methodologies for purposes of describing these examples, but one of ordinary skill in the art can recognize that many further combinations and permutations of the present embodiments are possible. Accordingly, the embodiments disclosed and/or claimed herein are intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

Computing devices typically comprise a variety of media, which can comprise computer-readable storage media and/or communications media, which two terms are used herein differently from one another as follows. Computer-readable storage media can be any available storage media that can be accessed by the computer and comprises both volatile and nonvolatile media, removable and non-removable media. By way of example, and not limitation, computer-readable storage media can be implemented in connection with any method or technology for storage of information such as computer-readable instructions, program modules, structured data or unstructured data. Computer-readable storage media can comprise the widest variety of storage media including tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

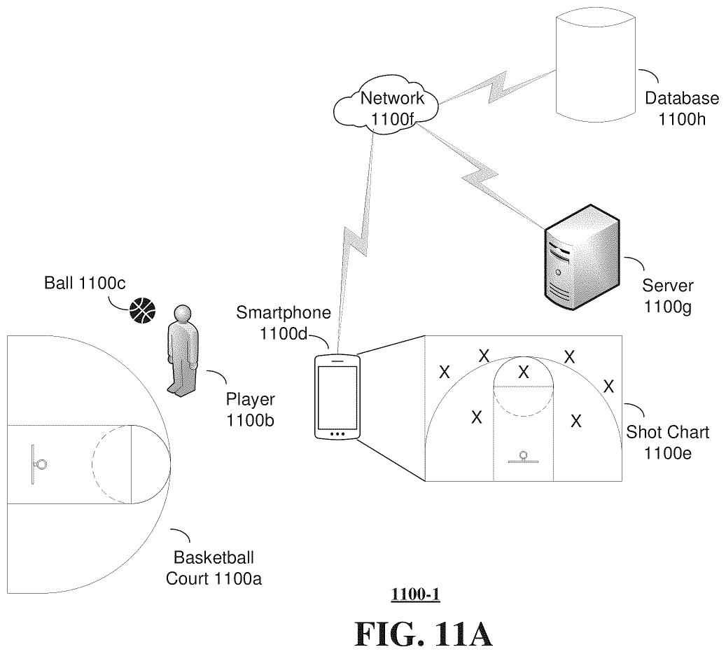

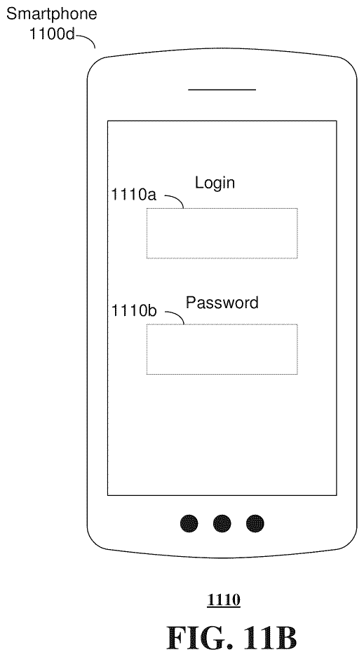

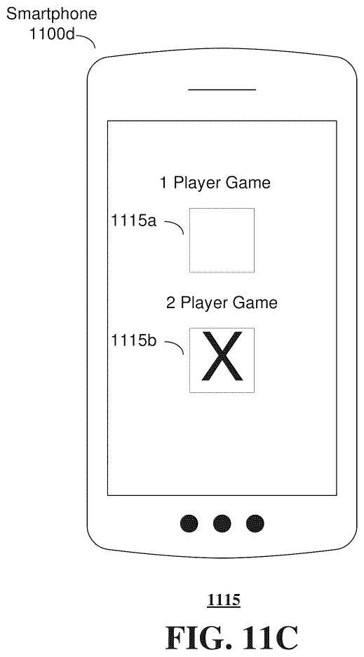

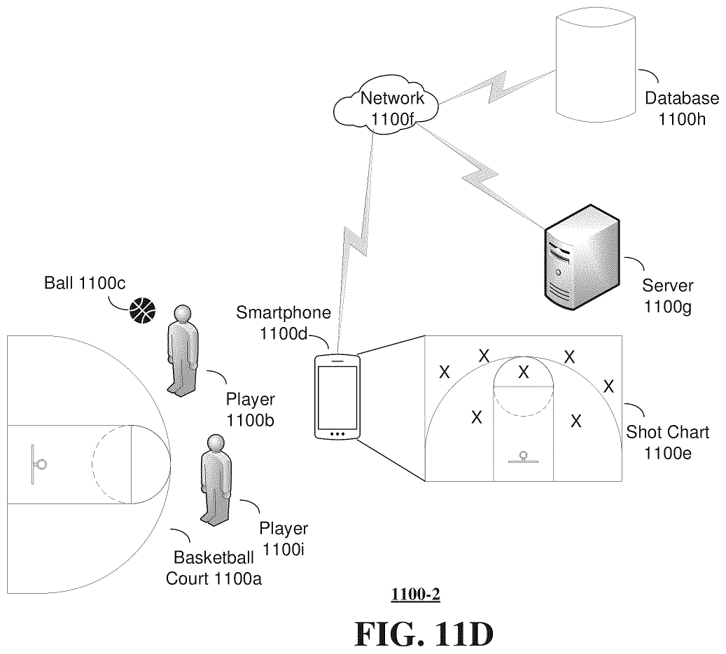

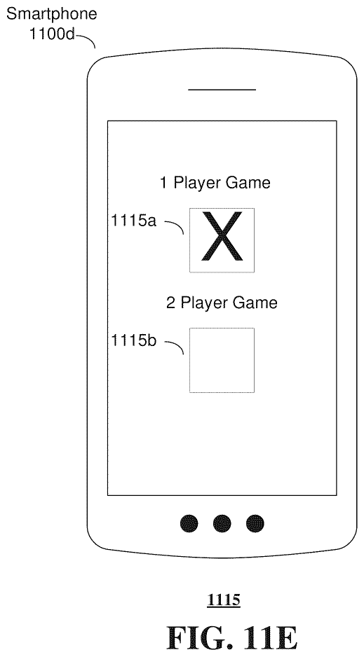

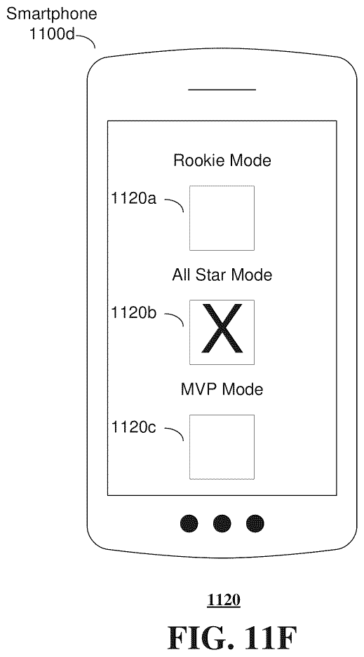

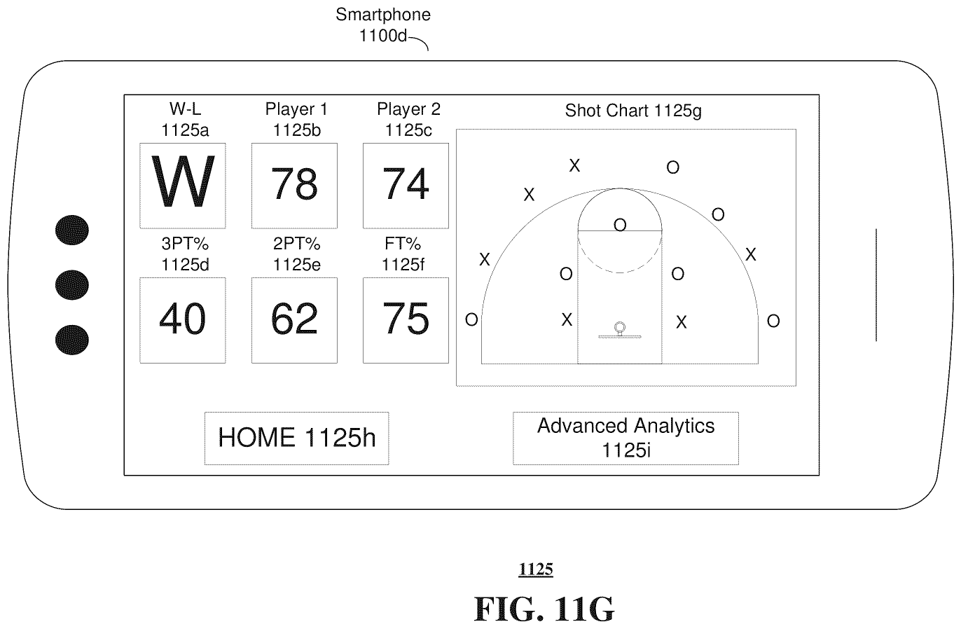

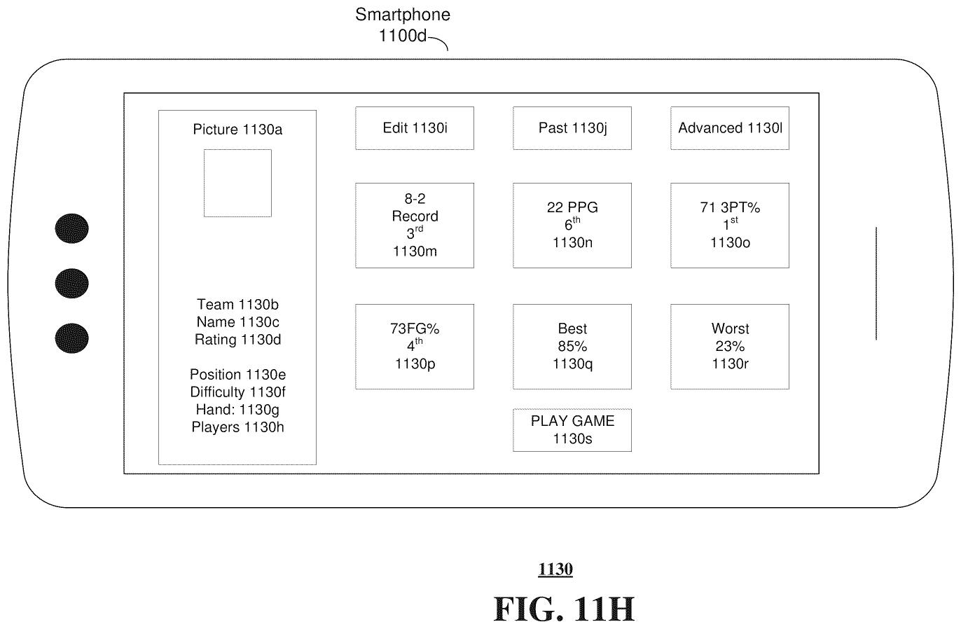

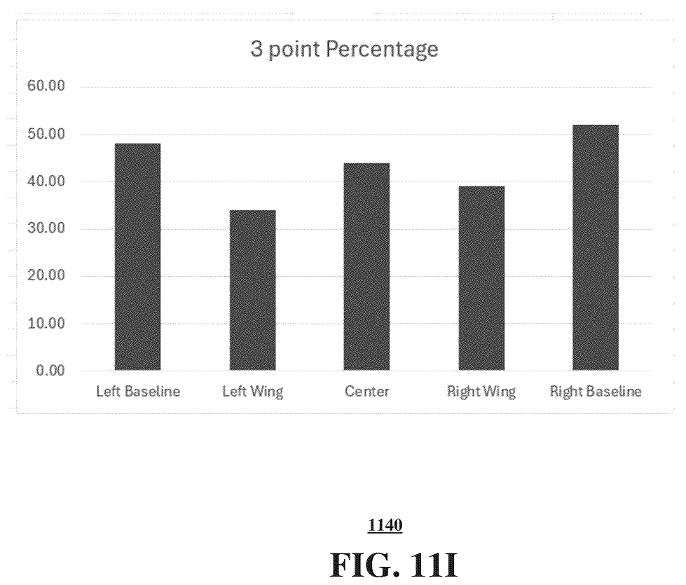

FIG. 11A, FIG. 11B, FIG. 11C, FIG. 11D, FIG. 11E, FIG. 11F, FIG. 11G, and FIG. 11I are block diagrams illustrating exemplary, non-limiting embodiments of a system to determine the biomechanics of a player to perform a skill in accordance with various aspects described herein. Referring to FIG. 11A, one or more embodiments include a system 1100-1 for providing a gamified basketball shooting training experience and collecting performance data for analysis.

In one or more embodiments, the system 1100-1 can operate exclusively or partially within a setting of a basketball court 1100a on which a player 1100b attempts basketball shots using a ball 1100c. A smartphone 1100d (or other communication device) is positioned to support a gamified training session for the player 1100b, which can include recording video content of the player 1100b attempting each of the a group of shots in a shot chart 100e. This smartphone 1100d can be positioned on a stand or tripod as well as held by another person (coach, trainer, parent, etc.) such that it can record the player 1100b attempting each basketball shot and determining whether the player 1100b makes or misses the attempted basketball shot. In various embodiments, the smartphone 1100d executes or runs one or more gamification software applications that can present the player 1100b the shot chart 1100e. The shot chart 1100e can define, prescribe, or otherwise indicate a sequence and/or types of basketball shots to be attempted during a training session or game session (e.g., a simulated game, a challenge mode, or a season-style progression). In one or more embodiments, any number of smartphones 1100d can be utilized which are positioned at various locations and/or controlled by any number of users for collecting images or other information.

In one or more embodiments, the smartphone 1100d is communicatively coupled to a network 1100f, which provides connectivity to a server 1100g. The server 1100g can execute one or more software applications, including analytics and/or artificial intelligence routines, to support the gamified basketball shooting session, including generating or updating the shot chart 1100e, tracking shot outcomes, and generating player performance metrics. In some embodiments, the server 1100g is further communicatively coupled to a database 1100h that stores information used by the system 1100-1, such as player profiles, shot libraries, difficulty settings, historical shot results, season statistics, simulated opponent data, and/or other training and performance analytics data. In further embodiments, the functions of the server can be performed by the smartphone 1100d. In additional embodiments, server 1100g can comprise one or more servers residing in one location, one or more servers spanning multiple locations, one or more virtual servers residing in one location, one or more virtual servers spanning multiple locations or one or more cloud servers. In some embodiments, database 1100h can comprise one or more databases residing in one location, one or more databases spanning multiple locations, one or more virtual databases residing in one location, one or more virtual databases spanning multiple locations or one or more cloud databases. In further embodiments, network 1100f can comprise one or more wired communication networks, one or more wireless communication networks, or combination thereof. Although the smartphone 1100d can be used to record the player 1100b attempting the basketball shots from the shot chart 1100e, another communication device associated with a camera can be used (e.g., tablet computer) to record and executing the gamification software applications.

In one or more embodiments, the system 1100-1 enables the player 1100b to perform real-world basketball shots on the basketball court 1100a using the ball 1100c while interacting with a gamified training experience presented on the smartphone 1100d, including the shot chart 1100e, with session data communicated via the network 1100f to the server 1100g and stored and/or retrieved from the database 1100h for analytics, tracking, and ongoing improvement of the training experience.

Referring to FIG. 11B, one or more embodiments include a system 1110 for user onboarding and authentication for a gamified basketball shooting training application executed by a smartphone 1100d, in accordance with various aspects described herein.

In one or more embodiments, the system 1110 is implemented at least in part by the smartphone 1100d, which executes one or more software applications (including a gamification application and, in some embodiments, an AI software application) to enable a user to access basketball shooting game sessions and associated performance tracking features. The system 1110 provides an authentication graphical user interface (GUI) that includes a login dialog box 1110a as well as a password dialog box 1110b, enabling secure access to a player account and associated data regarding the basketball shooting game sessions.

In operation, the smartphone 1100d can present, via a GUI, the login dialog box 1110a to receive user credential information (e.g., an email address or username) and the password dialog box 1110b to receive a corresponding password. Upon receiving the login 1110a and password 1110b, the smartphone 1100d can authenticate the user locally and/or via a remote service (e.g., a server/database as described with respect to FIG. 11A), thereby enabling access to the gamified basketball shooting game sessions, player profile information, and stored performance metrics for the authenticated player.

Referring to FIG. 11C, one or more embodiments can include a system 1115 for initiating and selecting a gameplay mode for a gamified basketball shooting training application executed by a smartphone 1100d, in accordance with various aspects described herein.

In one or more embodiments, the system 1115 is implemented at least in part by the smartphone 1100d, which executes a group of software applications (including a gamification/training software application and, in some embodiments, an AI software application) to generate and conduct basketball shooting game sessions. The system 1115 provides a mode-selection interface via which a user can select between a one player game 1115a and a two player game 1115b. In some embodiments, selection of a one player game or a two player game can be done by checking a corresponding dialog box, for example checking a dialog box for a two player game.

In operation, the smartphone 1100d presents, via a GUI, a selectable option corresponding to the one player game 1115a. Selection of the one player game 1115a can cause the smartphone 1100d to generate a basketball shooting game session for a first player, present shot prompts for the first player to attempt, record video content of the first player attempting the prompted shots, analyze the recorded video content (e.g., by an AI software application), determine shooting metrics for the first player, and present the shooting metrics and game results on the smartphone 1100d, consistent with the game-session flow described herein. Further, a computer-generated player can be simulated to play against the first player. This computer-generated player can be based a real-life player. Scores are tracked to determine a winner between the first player and the computer-generated player for the game session, consistent with the competitive scoring embodiments described herein.

The smartphone 1100d further presents a selectable option corresponding to the two player game 1115b. Selection of the two player game 1115b can cause the smartphone 1100d to generate a basketball shooting game session in which shot prompts are presented for a first player and a second player, video content is recorded for each player's attempts, shooting metrics are determined for each player, and scores are tracked to determine a winner between the first player and the second player for the game session, consistent with the competitive scoring embodiments described herein.

Referring to FIG. 11D, in one or more embodiments, system 1100-2 is similar to system 1100-1 but includes a second player 1100i that is competing with a first player 1100b in a gamified basketball shooting game session. Further, system 1100-2 conducts the gamified basketball shooting game session using smartphone 1100d and networked analytics resources, in accordance with various aspects described herein and consistent with the system of FIG. 11A and the mode-selection functionality described with respect to FIG. 11C.

In one or more embodiments, the system 1100-2 is deployed at a basketball court 1100a where a player 1100b attempts basketball shots using a ball 1100c. A smartphone 1100d executes a gamification software application (and, in some embodiments, an AI software application) that facilitates a selected gameplay mode (e.g., a one player game or a two player game as described with respect to FIG. 11C) and presents a shot chart 1100e to the player 1100b and to player 1100i. The shot chart 1100e can define, prescribe, or otherwise indicate a sequence and/or types of shots to be attempted during a basketball shooting game session, such as a simulated game, challenge mode, or season-style progression.

In one or more embodiments, the smartphone 1100d captures session data associated with the shot chart 1100e, including, for example, recording video content of the player 1100b attempting shots corresponding to the shot chart 1100e and/or receiving outcome inputs (manual or automatic) indicating made and missed shots, consistent with the game-session generation, video capture, and metric-determination operations.

The smartphone 1100d is communicatively coupled to a network 1100f, which provides connectivity to a server 1100g. In various embodiments, the server 1100g supports the gamified basketball shooting game session by performing one or more operations including (i) generating and/or updating the shot chart 1100e, (ii) determining or refining shot prompts based on difficulty, position, and/or game context, and/or (iii) analyzing captured information (e.g., video content and/or outcome inputs) to determine shooting metrics and game results. In some embodiments, the server 1100g stores and retrieves information from a database 1100h, including player profiles, shot libraries, prior performance history, season records, opponent parameters, and analytics outputs used to personalize and improve subsequent basketball shooting game sessions.

In one or more embodiments, the system 1100-2 enables the player 1100b to perform real-world basketball shots on the basketball court 1100a using the ball 1100c while participating in a gamified basketball shooting game session presented on the smartphone 1100d, including the shot chart 1100e, with data and analytics communicated via the network 1100f to the server 1100g and stored and/or retrieved from the database 1100h to support gameplay logic, scoring, metric generation, and progression.

Referring to FIG. 11E, one or more embodiments can comprise a system 1115, which includes a smartphone 1100d implementing a gameplay mode selection interface for initiating a gamified basketball shooting game session, in accordance with various aspects described herein.

In one or more embodiments, the system 1115 is implemented by the smartphone 1100d executing a group of software applications (including a gamification software application and, in some embodiments, an AI software application). The system 1115 presents, on a display associated with the smartphone 1100d, selectable gameplay-mode options including a one player game 1115a and a two player game 1115b.

In operation, selection of the one player game 1115a causes the smartphone 1100d to generate and conduct a basketball shooting game session for a first player, including presenting shot prompts (e.g., via a shot chart), recording video content of shot attempts via a camera of the smartphone 1100d, determining shooting metrics for the first player based on the recorded video content, and presenting game results and performance metrics, consistent with the game-session generation described herein. In some embodiments, a user can select a one player game by checking dialog box 1115a.

Selection of the one player game causes the smartphone 1100d to generate and conduct a basketball shooting game session for one player playing against a computer-generated player, including presenting shot prompts for the player, recording video content of the player's shot attempts, determining shooting metrics for the player, and tracking scoring to determine a winner between the player and the computer-generated player, the competitive session flow described herein.

In one or more embodiments, the smartphone 1100d, via the system 1115, supports initiating either the one player game 1115a or the two player game 1115b, thereby enabling different gamified basketball shooting session configurations while maintaining the common architecture of shot prompting, video capture, AI-driven analysis, metric determination, and results presentation.

Referring to FIG. 11F, one or more embodiments can include a system 1120 for selecting a difficulty level for a gamified basketball shooting training application executed by a smartphone 1100d, in accordance with various aspects described herein.

In one or more embodiments, the system 1120 is implemented at least in part by the smartphone 1100d, which executes a group of software applications (including a gamification software application and, in some embodiments, an AI software application) to generate and conduct basketball shooting game sessions. The system 1120 provides a difficulty-selection interface via which a user can select among a plurality of difficulty levels that affect shot prompting, simulated opponent skill, and/or scoring logic for the basketball shooting game sessions, consistent with the disclosure describing tiered shot prompts and difficulty-dependent game behavior.

In the illustrated embodiment, the system 1120 presents selectable difficulty modes including rookie mode 1120a, all star mode 1120b, and MVP mode 1120c. Selection of rookie mode 1120a can cause the smartphone 1100d to generate and present basketball shot prompts selected from a lower-difficulty portion of a shot database and to conduct a basketball shooting game session in which simulated gameplay conditions (e.g., opponent scoring probabilities and/or shot types) are configured for an entry-level experience. Selection of all star mode 1120b can cause the smartphone 1100d to generate and present basketball shot prompts selected from a higher-difficulty portion of the shot database and to conduct the basketball shooting game session with more challenging simulated gameplay conditions. Selection of MVP mode 1120c can cause the smartphone 1100d to generate and present basketball shot prompts selected from an advanced portion of the shot database and to conduct the basketball shooting game session with the highest difficulty simulated gameplay conditions. Note, the different difficulty modes can be selected in both one player mode and two player mode.

In one or more embodiments, based on the selected difficulty mode (e.g., rookie mode 1120a, all star mode 1120b, or MVP mode 1120c), the smartphone 1100d generates a group of basketball shooting game sessions, presents corresponding groups of basketball shots for a player to attempt, records video content of the player attempting the shots, analyzes the recorded video content, determines shooting metrics, and presents the shooting metrics, consistent with the operations recited herein. In some embodiments that include a computer-generated opponent, the selected difficulty mode can further configure the opponent simulation, such as by adjusting scoring probabilities and/or shot selection logic for the opponent.

In one or more embodiments, the system 1120 enables the smartphone 1100d to receive user selection of a difficulty mode (e.g., rookie mode 1120a, all star mode 1120b, or MVP mode 1120c) and to conduct gamified basketball shooting game sessions in accordance with the selected difficulty level.

Referring to FIG. 11G, in one or more embodiments, system 1125 presents results and performance metrics for a gamified basketball shooting game session on a smartphone 1100d, in accordance with various aspects described herein.

In one or more embodiments, the system 1125 is implemented at least in part by the smartphone 1100d executing a group of software applications (including a gamification software application and, in some embodiments, an AI software application) that (i) tracks shot outcomes for one or more players during a basketball shooting game session, (ii) determines shooting metrics based on recorded video content and/or user input, and (iii) presents game results and statistics via a graphical user interface.

In the illustrated embodiment, the system 1125 presents a win or loss indication 1125a corresponding to an outcome of a basketball shooting game session. In some embodiments, the win or loss indication 1125a is determined based on a comparison of scores tracked for two competitors (e.g., two human players, or a human player versus a computer-generated player), consistent with the scoring and winner-determination operations.

In one or more embodiments, the system 1125 presents a player 1 score 1125b and a player 2 score 1125c, each score corresponding to made shots attributed to a respective player during the basketball shooting game session. In various embodiments, the player 1 score 1125b corresponds to a first player associated with recorded video content captured by a camera of the smartphone 1100d, and the player 2 score 1125c corresponds to a second player (or a computer-generated opponent) as described herein.

The system 1125 further presents shooting-percentage metrics derived from the tracked shot outcomes and/or determined shooting metrics, including a three point percentage 1125d, a two point percentage 1125e, and a free throw percentage 1125f. In one or more embodiments, these percentages are computed based on attempts and makes for each shot category during the basketball shooting game session, and can be stored as part of a player profile and/or season statistics for subsequent basketball shooting game sessions.

In addition, the system 1125 presents a shot chart 1125g indicating locations and/or outcomes of shots attempted during the basketball shooting game session. In some embodiments, the shot chart 1125g is populated based on shot prompts presented during gameplay and corresponding make/miss outcomes determined from video analysis, manual input, or other outcome-detection mechanisms as described herein.

In one or more embodiments, the system 1125 includes navigation controls presented on the smartphone 1100d, including a home page push button 1125h selectable to return to a home screen or main menu of the gamification application, and an advanced analytics push button 1125i selectable to access additional performance analytics. In one or more embodiments, selection of the advanced analytics push button 1125i causes presentation of further statistics and/or AI-generated insights (e.g., trends, breakdowns by shot type or court region, or other derived metrics) based on stored session data.

Referring to FIG. 11H, in one or more embodiments, system 1130 for presents a player profile and performance-summary user interface for a gamified basketball shooting training application on a smartphone 1100d, in accordance with various aspects described herein.

In one or more embodiments, the system 1130 is implemented at least in part by the smartphone 1100d executing a group of software applications (including a gamification software application and, in some embodiments, an AI software application). The system 1130 can present player identity information, player-configuration information, and performance statistics derived from one or more basketball shooting game sessions (including, in some embodiments, a season of games).

In the illustrated embodiment, the system 1130 presents a player picture 1130a associated with a player profile. The system 1130 further presents a team name 1130b and a player name 1130c. The system 1130 also presents a rating 1130d, which in some embodiments represents an overall player rating determined based on aggregated shooting metrics (e.g., accuracy, scoring, and/or other session statistics). The system 1130 further presents a position 1130e (e.g., guard or forward) and a selected difficulty 1130f (e.g., rookie, all-star, MVP), each of which can influence shot selection, shot prompting logic, and/or simulated opponent behavior for game sessions. The system 1130 also presents a hand 1130g indicating a player handedness attribute used in configuring shot prompts and/or analysis.

The system 1130 further includes controls to manage and navigate player information and historical sessions. For example, the system 1130 includes an edit button 1130i selectable to edit one or more fields of the player profile (e.g., player picture 1130a, team name 1130b, player name 1130c, position 1130e, difficulty 1130f, and/or hand 1130g). The system 1130 further includes a past button 1130j selectable to access previously completed game sessions and/or historical performance results for the player.

In one or more embodiments, the system 1130 further includes an advanced analytics button 11301 selectable to access additional analytics generated from recorded video content and/or determined shooting metrics, such as breakdowns by shot type, court location, time-based trends, streak behavior, or other performance insights described in the disclosure.

In the illustrated embodiment, the system 1130 presents a record 1130m indicative of a win/loss record or other aggregated record for a set of basketball shooting game sessions (e.g., across a season). The system 1130 also presents session-derived or season-derived statistics including points per game 1130n, three point percentage 1130o, and field goal percentage 1130p. The system 1130 can further present comparative performance indicators including a best field goal percentage 1130q and a worst field goal percentage 1130r, which can correspond to best and worst session performances over a defined set of game sessions or time period.

The system 1130 further presents a play game button 1130s selectable to initiate a new gamified basketball shooting game session or to continue a current game session. In response to selection of the play game button 1130s, the smartphone 1100d can generate a basketball shooting game session, present shot prompts for a player to attempt, record video content of the player attempting the shots via a camera of the smartphone 1100d, analyze the recorded video content using an AI software application, determine shooting metrics, and present results, consistent with the operations recited herein.

Referring to FIG. 11I, one or embodiments can show advanced analytics such as three point percentage based court location as a bar chart 1140.

One or more embodiments can operate as a mobile-based, gamified basketball shooting simulator implemented on a smartphone, in which audio prompting, user input, and game-state updates are coordinated to make an individual shooting workout operate like a structured basketball game. In some embodiments, the application can begin with a profile setup flow in which the user signs in and configures parameters such as a selected team, player name, jersey number, and a difficulty level. The difficulty level operates as a control input that affects the game logic, including the types of shots that will be generated and the performance threshold needed to win a simulated game. In other embodiments, after profile configuration the application can provide a home screen that is configured to track progression over multiple games, such as win/loss results and standings, thereby framing repeated practice sessions as an ongoing season-style experience rather than isolated drills.

One or more embodiments implements a prompt-and-response gameplay loop that supports hands-free or reduced-friction operation using wireless headphones. The smartphone outputs shot prompts as audible instructions telling the user what shot to take next, and after each attempt the user provides an outcome input to the application on the smartphone indicating “make” or “miss.” Further embodiments support different outcome entries including voice input through wireless headphones and manual input by touching the smartphone screen, allowing the same game state engine (e.g., software application) to operate even when voice input is unavailable. Voice input improves usability on a court because it reduces the need for the user to repeatedly return to the device between shot attempts.

One or more embodiments include an implementation in which shot outcomes are determined automatically using the smartphone camera and computer vision, rather than relying exclusively on user generated input outcomes (e.g., voice input, manual input etc.). Automatic detection of a “make” or “miss” depends on stable camera placement and an adequate field of view (e.g., visibility of the three-point arc) to reliably detect shot attempts and results. In another embodiment, the gameplay loop incorporates timing between prompts, such that the user has an interval between shots that can be used for movement or conditioning activities, which helps approximate the cadence of possessions in a real game while also increasing the physical training load.

One or more embodiments increase real game situations and immersion by combining shot prompts with game commentary and score updates, such as announcing a virtual location, indicating the start of a quarter, issuing detailed multi-step shot instructions tied to basketball court regions and movement sequences, and then updating the scoreboard after the outcome is registered. Technically, this can be implemented as a state-driven sequence in which each prompted attempt triggers an outcome registration event, the event updates the simulated score and game state, and the updated state determines the next prompt and narration/commentary. In this manner, the smartphone-based application operates as an interactive simulation engine (e.g., software application) that orchestrates prompted physical actions, records or receives outcomes, and presents an evolving game narrative and performance feedback to the user during the training session.

One or embodiments include a profile and player-configuration flow implemented by the mobile application on the smartphone, where a user logs in and selects a team, position, handedness, and difficulty level. The team selection can reference a fictional league or real-life league (e.g., NCAA, NBA, or other professional teams). The position selection (e.g., guard, wing, center) is used as an input parameter to tailor which shots and actions are prompted during gameplay, such that guards may receive more off-the-dribble actions while centers may receive more post-oriented actions. The handedness selection is similarly used to tailor prompts (e.g., shot selection) and expected movements, such as differentiating right-hand versus left-hand drive and finish behavior, and the difficulty level selection (e.g., rookie, starter, all-star, MVP). further, the difficulty level can be used to adjust shot difficulty, the distribution of easy versus difficult shot prompts, and the performance demands needed to win against the computer-generated opponent.

One or more embodiments include capturing shot outcome information using either manual or automated mechanisms. A manual implementation supports registering makes and misses through voice input via wireless headphones or by the user inputting make/miss controls on the mobile application on the smartphone, thereby enabling play even when automatic sensing is not available. A further embodiment adds automatic sensing make/miss tracking using the smartphone camera and computer vision, enabling real-time determination of shot outcomes without requiring user declaration. To reduce user/player cheating and to validate that the user/player attempted the prompted shot type and location, further embodiments include distance tracking and/or area tracking on the basketball court. In these embodiments, if the mobile application prompts a three-point shot from a specific zone (e.g., right corner) but the player instead attempts a shot from a materially different zone or distance, the mobile application can detect the mismatch, via the smartphone camera and computer vision, and can record the attempt as a miss or otherwise penalize the result, thereby enforcing adherence to the prompted training regimen.

One or more embodiments include an “improve” or training component to the competitive gameplay so that the mobile application not only simulates a game but also gathers movement and performance data that can be correlated to success rates. These embodiments can include measuring or estimating biomechanic and/or movement-pattern metrics during shot attempts and linking those metrics to whether shots were made or missed. By combining training-game data with improvement analytics, one or more embodiments can build individualized performance models that identify movement patterns associated with better outcomes for a particular player, which can then be used to generate feedback during or after gameplay and to influence future training recommendations or generate training exercises, drills, etc.

One or more embodiments record, aggregate, and present detailed statistics and analytics across games to support player development. This includes tracking conventional metrics such as points per game, shooting percentages, and win/loss records, and also generating shot charts that visually map makes and misses to basketball court regions based on a shot database and zone mapping. The analytics are organized at multiple levels of granularity, including identifying best and worst shot types (e.g., layups versus three-pointers), best and worst specific shots (e.g., subcategories of a shot type) within a shot type (e.g., step-back three versus catch-and-shoot three), and performance across combined subcategories that reflect more detailed context (e.g., a left-hand dribble into a three-point shot). In this embodiment, the mobile application uses the accumulated performance data to prescribe targeted drills and workouts that address demonstrated weaknesses, thereby converting gameplay-derived performance measurements into personalized training actions.

One or more embodiments include a shot database implemented as a structured collection of shot definitions from which the mobile application selects and presents shot prompts during gamified basketball shooting game sessions. The shot database operates as a configurable source that generates available shots by difficulty level, enabling the mobile application to pull different sets of shots depending on whether the player is in a lower difficulty mode (e.g., rookie) or a higher difficulty mode (e.g., MVP). In such embodiments, when the mobile application determines a target probability or deterministic distribution of shot types for a game session such as how many layups, mid-range attempts, or three-point attempts the player is to be prompted to take, it can randomly select specific shot instances from the shot database that satisfy that probability or deterministic distribution. In some embodiments, the randomization can be constrained to ensure coverage over time, such as ensuring that during a season of games the player is prompted with a variety of different layup types or other shot variations rather than repeatedly receiving the same prompts.

In another embodiment, each shot definition in the shot database includes fields that support both gameplay prompting and subsequent analytics. For example, shot entries are categorized by shot type and one or more subcategories that describe contextual attributes of the shot, such as dribble-hand, movement pattern, action type (e.g., step-back, pick-and-roll, off-the-catch), or other descriptors. These structured fields enable the system to analyze performance at different granularities, including aggregate performance by shot type and more detailed performance by combinations of subcategories, thereby supporting the player development and analytics functions described elsewhere. In addition, the shot database can include metadata indicating which shots are applicable based on a player's position and handedness, allowing the mobile application to tailor the prompted shots to the player profile so that prompts better match expected real-world tendencies and training needs.

In a further embodiment, the mobile application includes a shot chart mapping that associates each prompted shot to a defined court zone so that makes and misses can be visualized after the game as a shot chart. In this embodiment, each shot entry is associated with a zone identifier (e.g., an integer code) that corresponds to a location on a zone map. The zone map defines discrete court regions such as corners, wings, top of the key, mid-post, paint areas, and other locations, and the zone identifier serves as the key between a prompted shot and a position on the rendered shot chart. As a result, when the user/player completes a game session and the mobile application records an outcome for each prompted shot, the mobile application can generate a visual representation via the shot chart showing where shots were attempted and whether they were made or missed.

In another embodiment, the mobile application supports iterative expansion and revision of the shot database and zone mapping to improve accuracy and to add additional shots, subcategories, and court locations. This embodiment can include migrating to a different shot database framework in which shot definitions are more comprehensive and structured, enabling more precise prompting, more detailed analytics, and a more informative shot chart. Through this database-driven approach, the application separates the content layer (shot definitions and metadata) from the game logic layer (how shots are selected and sequenced), which allows updates to shot offerings, difficulty tuning, and zone mapping without requiring fundamental redesign of the core gameplay engine (e.g., software application).

In one or more embodiments, the mobile application simulates a basketball game by normalizing the number of shot opportunities so that the experience is understandable and balanced within a training context. Rather than attempting to model every source of shot-count variance found in real games, such as turnovers and offensive rebounds, the mobile application can allocate a fixed number of total shots to each side during a scripted portion of gameplay. For example, the opponent is assigned 60 shots and the user side is also assigned 60 shots for the first three quarters and the first ten minutes of the fourth quarter. This fixed-shot allocation embodiment creates a controlled scoring environment in which the user can interpret the scoreboard and outcome as being driven primarily by shooting performance rather than by simulated randomness in possession count.

In another embodiment, the mobile application partitions the user side's total shot allocation between shots physically attempted by the user and shots attributed to the user's simulated (e.g., computer-generated) teammates. For example, out of 60 total user-side shots, the user may be prompted to take 13 shots while the user's team takes the remaining 47 shots. This partitioning creates the feel of participating as a single player within a larger team game while preserving the user's influence on the outcome. If the simulated opponent and simulated teammates perform at comparable levels to one another, the user's efficiency on their prompted attempts becomes determinative whether the user's team is ahead or behind entering the final phase of the game session.

In a further embodiment, the mobile application controls the ordering of possessions by interleaving user attempts with simulated possessions by the opponent and the user's team to maintain realistic score/game progression. The user may take the first shot, after which the system alternates simulated possessions between the opponent and the user's team for several possessions before returning to the user for the next prompted shot. This possession-spacing embodiment ensures the score changes in plausible increments, avoiding unrealistic jumps that would occur if too many simulated possessions were done between user attempts. Similar embodiments also support distributing the user's 13 prompted shots across quarters. For example, a greater number early and fewer in later quarters or a roughly even distribution so that the user's participation is temporally spread across the game in a manner consistent with typical gameplay pacing rather than being concentrated in a single segment.

In another embodiment, the mobile application distinguishes between a scripted portion of gameplay and a late-game portion governed by separate game logic. The fixed-shot, evenly distributed flow can apply, for example, only through the first three quarters and ten minutes, after which a late-game logic embodiment governs the final two minutes of simulated time. This separation allows the mobile application to transition from a predetermined or tightly controlled scoring flow to an end-game sequence that can place greater onus on the user, increase back-and-forth engagement, and support time-and-score-sensitive behavior, thereby emphasizing “clutch” play and enhancing the realism and motivational qualities of the training simulation.

In one or more embodiments, the mobile application defines game logic by specifying shot totals and shooting percentages for the opponent, the user, and the user's team, and then using those parameters to produce a realistic simulated score environment during a game session that the user can influence through their own prompted attempts. For example, in an all-star mode configuration, the opponent is allocated 60 total shot events, with an acknowledgment that a subset may represent grouped free-throw events (e.g., three “sets” corresponding to multiple free throws), and those 60 events are distributed across shot types such as layups, floaters, fadeaways, free throws, mid-range jump shots, and three-point jump shots. In this embodiment, each shot type is paired with an intended opponent make-rate (e.g., high percentages for layups and lower, but still strong, percentages for three-pointers), which yields an expected number of made shots per shot type and therefore an expected point total for the scripted portion of the game. The aim is for the opponent's score to land around a target value (e.g., approximately 100 points) in the first three quarters and ten minutes, creating a consistent difficulty level.

In another embodiment, the mobile application ensures a balanced playing by matching the total shot count and shot-type mix of the user side to that of the opponent, while dividing the user side's attempts between the user and simulated teammates. For example, the user and the user's team together are allocated the same 60 shot events as the opponent, but the user personally receives only 13 shot events while the simulated teammates receive the remaining 47 shot events. This partitioning creates a gameplay structure in which the user's team contributes a predictable baseline score and the user's own performance must make up the differential required to win. In this embodiment, if the user's team is modeled to score a defined number of points on its 47 shots, the user's 13 possessions become the decisive factor. For example, if the opponent is expected to score about 100 points and the user's team contributes a fixed portion, the user must score enough points on their limited possessions to reach even or take a lead heading into the late-game sequence.

In a further embodiment, the mobile application introduces controlled variance so that opponent scoring is not identical from game to game while still remaining within reasonable bounds. Rather than performing a fully random “coin flip” on every opponent shot throughout the entire game, which can lead to extreme outcomes such as unrealistically high three-point success, the mobile application assigns a range of possible made-shot totals for each shot type and uses weighted probabilities or different probability distributions with different mean and variances (e.g., normal distribution, uniform distribution, Pareto distribution, binomial distribution, etc.) to select outcomes within that range for each game. For example, the opponent may have a minimum and maximum number of makes for layups, floaters, mid-range shots, and three-pointers, with a higher probability assigned to an average outcome and lower probabilities assigned to the extremes. This variance is tuned to keep the opponent's score within a narrow band around the target (e.g., roughly 95 to 105 points in the all-star configuration), thereby preserving real game scenarios while maintaining a stable difficulty curve and avoiding outlier games that feel unfair or implausible. In some embodiments, additional variance can be introduced on the user-side team simulation as well, which primarily applies variance to the opponent to keep the user experience engaging without destabilizing the core competitive balance.

In one or more embodiments, the mobile application divides gameplay into a regular game portion and a late-game portion to balance real life game situations with controllability. For example, during the first three quarters and the first ten minutes of the fourth quarter, the opponent's scoring is largely determined before the game begins using predetermined totals, subject to limited variance, so the opponent might be assigned a specific number of made shots out of a fixed number of total shots for that session. This predetermined scoring embodiment also includes an “even distribution” mechanism so that the opponent's assigned makes are not clustered into a single quarter but are instead spread across the game in a way that produces plausible score progression. The purpose of this structure is to avoid the instability that would occur if the mobile application performed a probabilistic coin-flip determination for every opponent attempt across the entire game, which can cause skewed outcomes such as the opponent converting an unrealistic proportion of three-point shots.

In one or more embodiments, the mobile application switches into a late-game logic mode during the final segment of the game, such as the last two minutes, where the sequence of possessions changes to emphasize user agency and clutch performance. In these late-game logic embodiments, the simulated “user team” possessions are removed and the possession order becomes a direct back-and-forth between the user and the opponent, increasing the user's responsibility for the outcome. Additionally, opponent outcomes in the late-game segment are no longer predetermined; instead, each opponent attempt is based on using a probability-based distributions tied to the opponent's shooting percentages for the selected difficulty level. For example, if the opponent's configured three-point percentage in an all-star mode is 65%, then when the opponent is prompted to take a three in late game, the mobile application resolves that attempt using a randomized selection process (e.g., probability distribution with mean and variance) consistent with a 65% make mean probability, while a mid-range attempt might be resolved using a different probability value defined for that shot type.

In one or more embodiments, late-game shot prompting can be based on time and score to replicate real basketball decision-making. The mobile application can determine the remaining time and current score differential to determine which shots are eligible to be prompted, and can constrain the selection of the shots by shot type accordingly. For example, if the user is down by three points with only a few seconds remaining, the mobile application can exclude non-three-point options from the shot database and prompt only a three-point shot; similarly, if the opponent is down by three late, the opponent is prompted to attempt a three point shot. This embodiment can also incorporate selectable or randomized shot choices within the constrained category, such as choosing among different three-point shot types or locations, while preserving the principle that end-of-game prompts should align with the strategic needs implied by the score and remaining time.

In one or more embodiments, gamification gameplay (e.g., the basketball shooting game session) are based on percentages and more random with some extra possessions included where the user's team can get an offensive rebound and an extra shot or late in the game the opponent may turn it over and the user gets an extra possession or even vice versa. In other embodiments, the game sessions work as the late game portions described herein where the opponents' shots are based on the percentages (e.g., one or more probability distributions). In further embodiments, the number of shots for both the user and/or the opponent (real player or computer generated) may also be governed by one or more probability distributions with some variance.

In one or more embodiments, the mobile application incorporates variance to prevent the simulated opponent from producing the same score in every game while still keeping outcomes within a realistic and controlled range. Rather than deterministically assigning identical made-shot totals for each shot type in each session, the mobile application determines, for each shot type in a given difficulty mode, a minimum made-shot value, a maximum made-shot value, and an average made-shot value. Such embodiments apply a fixed shot distribution for the opponent (for example, in an all-star configuration the opponent might be allocated a defined number of layups, floaters, fadeaways, free-throw sets, mid-range shots, and three-point shots out of a total such as 60), and the mobile application selects how many of each are made in a particular game by randomly choosing among the minimum, average, and maximum outcomes using weighted probabilities and/or probability distributions. For example, the mobile application can assign a 10% probability to the minimum outcome, a 10% probability to the maximum outcome, and an 80% probability to the average outcome, thereby producing most games near the expected score while occasionally producing modestly higher or lower opponent scoring.

In one or more embodiments, the variance is constrained by guard rails to ensure that the opponent's total points do not deviate into implausible extremes that would undermine fairness and user engagement. The target is to have the opponent's scoring land in a narrow band around an expected value based on the difficulty mode, such as a range roughly centered near 100 points in an all-star mode, rather than producing extremely low or extremely high totals that would provide a diminished user experience. Such embodiments ensure that even when per-shot-type variability is introduced, the overall game remains competitive and interpretable, and the user can attribute wins and losses primarily to their own execution rather than to wild random swings.

In one or more embodiments, the mobile application can optionally extend variance beyond the opponent and also vary the user-side shot environment, including the number of prompted user shots of a given type or the simulated performance of the user's team. For example, instead of prompting the user with exactly the same number of three-point attempts in every game, the mobile application could vary that count by game, such as prompting four, five, six, or seven three-point shots across different sessions. However, the mobile application can emphasize opponent variance as the primary variability mechanism while keeping the user's shot distribution more stable, because the late-game portion already introduces additional randomness driven by time and score logic. In this way, the mobile application serves to keep the simulation fresh and realistic while preserving a controlled difficulty experience and maintaining the user's sense of agency.

In one or more embodiments, the mobile application incorporates a concept of momentum that dynamically modifies simulated opponent performance in response to the user's shooting streaks, thereby better mimicking the psychological and statistical swings observed in real basketball. In such embodiments, the opponent's expected scoring for the regular game portion of the game may initially be determined using predetermined made-shot totals or predefined averages, but the mobile application allows those predetermined outcomes to be adjusted when the user exhibits “getting hot” behavior such as making multiple shots in a row. For example, if the user makes four, five, or six consecutive shots, particularly in a comeback scenario late in the game, the mobile application can reduce the opponent's predetermined scoring by removing points or subtracting one or more made shots that the opponent would otherwise have been credited with, thereby reflecting the idea that an opponent can tighten up under pressure when the other team gains momentum.

In one or more embodiments, momentum is implemented by altering the opponent's probability of making prompted shots during portions of the game where outcomes are resolved probabilistically, such as late-game play. In such embodiments, the mobile application maintains baseline shooting percentages for the opponent by shot type and difficulty level (for example, a defined three-point percentage in an all-star mode), and then applies a momentum modifier that reduces those percentages when the user is on a streak. For example, if the opponent's baseline probability of making a three-point shot is set to 65% in a given mode, and the user has just made three shots in a row late in the game, the system can reduce the opponent's effective three-point make probability to a lower value such as 45% for subsequent opponent attempts. This probability-adjustment embodiment allows momentum to influence the likelihood of opponent scoring without requiring fully deterministic manipulation, and it can be configured to apply selectively based on thresholds (e.g., streak length), game timing (e.g., late-game emphasis), or other contextual factors, thereby integrating user performance dynamics into the simulated competitive environment in a game session.

In one or more embodiments, the mobile application provides an audio-driven gameplay that simulates the feel of a live broadcast by combining shot prompting, game commentary, and periodic game-state updates. The audio output is structured around two distinct voices (e.g., tones), where a first voice functions as a shot prompt voice that instructs the player what shot to attempt and a second voice functions as a commentary voice that reacts to the outcome of the attempt and narrates the game. In such embodiments, the game sessions begins with an introductory announcement that communicates that play is underway and identifies a location and whether the user is conceptually home or away, followed by a quarter indicator (e.g., “first quarter”) so the user can track progression through the game without needing to look at the screen. The shot prompt voice then issues a specific instruction such as a shot location or shot type, the user attempts the shot, and the commentary voice provides immediate feedback such as “nice shot” or “no good,” which also serves as confirmation that the system registered a make or miss.

In one or more embodiments, the mobile application uses an audio transition effects (e.g., crowd noise, etc.) to simulate intervening possessions between user attempts and to keep the scoring flow plausible given that the user is only taking a subset of total game shots. After the commentary voice reacts to a user attempt, the system plays a fast-forward sound, crowd noise, or other transitional audio that signals that multiple simulated possessions occurred in the background. During or after this audio transition, the mobile application provides a score update that reflects the simulated scoring by the opponent and the user's team during those intervening possessions. This sequence repeats in a loop so that each prompted shot attempt by the user is bracketed by narration of game progress, including updated scores, which maintains immersion and allows the user to experience the session as a continuous game rather than a series of disconnected drills.

In one or more embodiments, the audio and commentary engine is coordinated with the gamification logic so that the mobile application can determine when to announce scores and when to suppress them for pacing and clarity. For example, the commentary voice may announce the score after a made shot but omit the score after a miss, while still confirming the miss, and then provide the next score update after the simulated possession transition. This enables consistent feedback, reinforces that the mobile application recognized the shot outcome, and communicates the evolving game state in a way that is understandable while the user is physically moving and shooting, particularly when the user is using headphones or a speaker and is not continuously viewing the display.

In one or more embodiments, the mobile application provides an integrated “show, teach, improve” capability that connects in-game shot prompting to on-demand instructional media so the player can understand and execute prompted shots and actions correctly. When the mobile application prompts a particular shot type, location, or basketball action that the player may not recognize, the mobile application can offer an in-game option to access a short video that shows what the shot looks like, where it occurs on the court, and how it should be performed. For example, the video may be based on game film or a demonstration clip of a coach or skilled player executing the same shot. This embodiment enables immediate clarification of terminology and positioning so that a user who is unfamiliar with a prompt can still participate in the gamified session without breaking the training flow.

In one or more embodiments, the mobile application provides a teaching mode that goes beyond visual demonstration by delivering technique-focused guidance associated with the prompted shot. In such embodiments, when a player selects a “teach” option within a teaching mode, the mobile application presents instructional content explaining key execution cues such as footwork, balance, follow-through, shot preparation, and situational reasoning for why the shot is used in real play and how defenses typically influence the shot choice. For example, for a transition three-point shot, the mobile application can teach pacing and footwork considerations, alignment and release cues, and decision-making context related to defensive recovery. This supports skill acquisition by pairing the prompted shot experience with targeted coaching content that can be accessed during gameplay or reviewed separately at any time.

In one or more embodiments, the mobile application implements an improvement engine (e.g. software application) that generates drills and workouts tailored to a player's measured weaknesses based on performance analytics accumulated across game sessions. The mobile application tracks makes and misses by shot type and by more granular subcategories, which allows it to detect patterns such as poor performance on a specific kind of three-point shot, or difficulty with shots taken while drifting or moving in a particular direction. Based on those detected weaknesses, the mobile recommends drills specifying quantities (e.g., number of attempts or makes), focal points, and structured repetitions designed to remediate the weakness. This turns gameplay-derived performance tracking into individualized training prescriptions rather than generic workout plans.

In one or more embodiments, the mobile application includes pregame drills and workouts that function as preparation before the user begins a simulated game, thereby controlling progression and introducing new basketball concepts in a structured way. In such embodiments, before a game session, the mobile application can require the player to complete a short set of drills that teach a new action, shot type, or tactical response, and then in the subsequent game prompts the system instructs the player to apply what was learned. For example, the mobile application can teach options for responding to different ball screen coverages, such as a trap, hedge, switch, or drop, and then prompt the player during the next game to execute shots or actions that correspond to those taught responses. This enables staged learning and ensures that more advanced prompts are introduced only after the player has been guided through preparatory instruction and practice.

In one or more embodiments, the mobile application provides configurable game options that determine how many participants are involved in a gamified basketball shooting session and whether the opponent is simulated or human. The default configuration can be a one-player experience in which a single user plays against a simulated opponent generated by the mobile application, with the mobile application tracking the user's makes and misses and updating the game score as the simulated opponent and the user's simulated team accumulate points. In another embodiment, the mobile application supports multiple players on the same side, such that two to five players can participate together on a shared court while still competing against a simulated opponent, with the mobile application coordinating shot prompts, attributing outcomes to the appropriate player, and maintaining game state and scoring for the group.

In a further embodiment, the mobile application supports remote competition against a real player in a different location. In this embodiment, two users can participate in a shared game session where each user takes turns attempting prompted shots at their respective courts, and the mobile application synchronizes scoring, game state, and progression between devices. The mobile application can optionally implement matchmaking so that a user is paired with an opponent of similar skill level, thereby improving fairness and engagement in head-to-head play.

In another embodiment, the mobile application supports in-person competition between real players at the same location. In this embodiment, two or more players can alternate shot attempts while using the mobile application to deliver prompts, record outcomes, and calculate the winner. The same embodiment can be extended to team formats, such as one-on-one, two-on-two, three-on-three, or larger groupings up to five-on-five, with the mobile application maintaining the rules for turn order, score updates, and result determination.

In yet another embodiment, the mobile application supports asynchronous competition in which players do not need to play at the same time. In this embodiment, a first player completes a game session and the mobile application stores the score and performance details, after which a second player later completes the same or a comparable game session, and the mobile application compares results to determine a winner. This asynchronous embodiment is analogous to turn-based mobile games in which participants play at different times, and it enables competitive engagement even when schedules or locations do not align.

In one embodiment, the mobile application provides multiple game modes that change the structure of the gamified basketball shooting experience, including how progression is tracked, how opponents are selected, and what off-court activities are integrated into gameplay. A season mode embodiment presents a sequence of games against a simulated opponent, where each game result contributes to an overall record and the mobile application advances the user through a season of a configurable number of games followed by a playoff format. For example, the mobile application can implement an 18-game season or a longer season such as 30 or 82 games, and can conduct playoffs using either a best-of-seven series format or a single-elimination format, with the objective of winning a championship.

In another embodiment, the mobile application provides a challenge mode in which the player begins at a lower difficulty level and progresses upward by meeting performance thresholds. In this embodiment, the mobile application gates advancement so that the user “works their way up” through increasingly difficult levels in a manner similar to video game progression, with shot prompts, opponent performance, or required shooting efficiency becoming more demanding as the user advances.

In a further embodiment, the mobile application provides a conditioning mode that integrates fitness or movement requirements between prompted shots. In this embodiment, after a shot prompt (or between shot attempts), the mobile application issues prompts requiring the user to perform conditioning activities such as sprints, defensive slides, push-ups, or other exercises, thereby combining skill training with conditioning and simulating the physical demands of a real game.

In another embodiment, the mobile application provides a live mode configured to facilitate matchmaking and real-time competition against other users. In this embodiment, the mobile application enables a user to enter a multiplayer environment, locate an opponent with a similar skill set or skill level, and then conduct a head-to-head session with synchronized prompts, scoring, and results, thereby creating an experience analogous to competitive video game matchmaking.

In yet another embodiment, the mobile application supports a team mode associated with a team subscription model that enables coaches and teams to deploy the application across multiple players with more role-specific configuration than a consumer mode. In this embodiment, instead of broad positions such as guard, wing, and center, the mobile application supports more granular positions tailored to team coaching needs, such as a shooting center, a pick-and-roll center, or a post-up center, and the mobile application selects or emphasizes shot prompts and actions aligned with those roles.

In a further embodiment, the mobile application incorporates pregame preparation and skill-development gating to introduce new basketball concepts and target weaknesses prior to gameplay. In this embodiment, as the player increases in level or progresses through modes, the mobile application introduces new shot types or actions such as coming off specific ball screens, and may require the player to complete drills or instructional segments before those actions are prompted in a game. For example, the mobile application can teach terminology and execution for actions like a double-drag ball screen and require completion of drills or workouts before prompting related shots in subsequent games, thereby ensuring that new concepts are learned and practiced before being evaluated under game-like conditions.

In one embodiment, the mobile application includes a gamification and rewards function configured to increase engagement by linking in-session performance and longer-term progression to unlockable achievements and benefits. In this embodiment, the mobile application monitors gameplay events such as win streaks, performance thresholds, and shot-based milestones, and then awards badges or other achievement indicators when the user satisfies corresponding criteria. For example, the mobile application can issue an award when the user reaches a defined win streak length, makes a specified number of three-point shots within a game, or wins a championship within a season mode, thereby associating training consistency and performance with recognizable accomplishments.

In another embodiment, the mobile application implements unlockable cosmetic or gameplay-affecting items that are granted based on achievement triggers. For example, the mobile application can unlock new virtual jerseys or other customization assets associated with the user's player profile, with the unlocked items existing in the mobile application environment rather than as physical goods. In a further embodiment, the mobile application can provide gameplay bonuses tied to achievements, such as granting an additional shot opportunity during a game when the user meets a predefined performance condition. This embodiment can be integrated with the game logic in which the user receives a limited number of prompted attempts during regulation, such that an “extra shot” reward increases the user's in-game agency and reinforces achievement-driven motivation.

In another embodiment, the mobile application supports prize-based incentives that extend beyond in-app rewards by enabling the awarding of real-world prizes. For example, when the mobile application detects that a user has met a reward condition, the mobile application can trigger eligibility for tangible benefits such as event tickets, sports equipment, or other items.

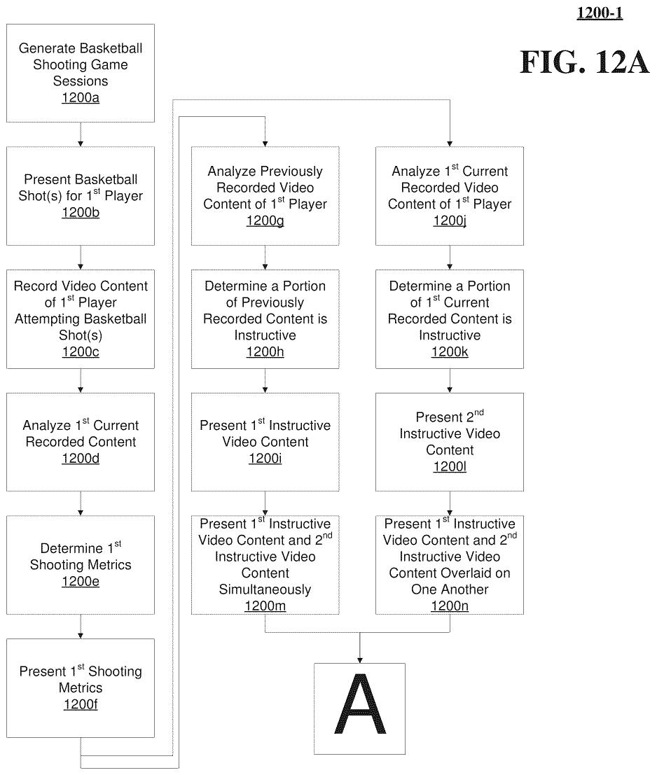

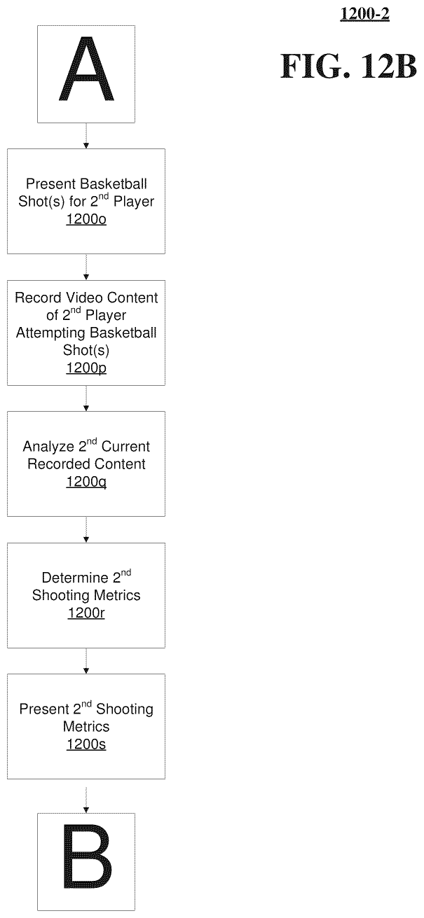

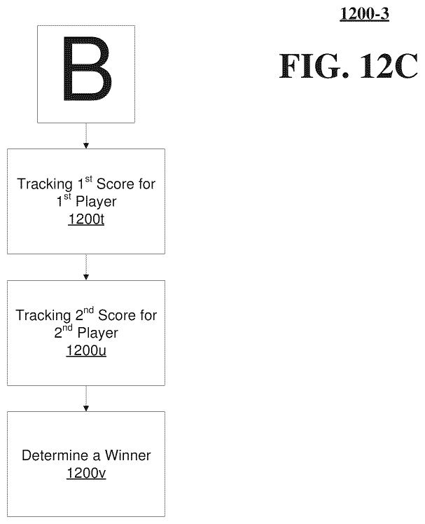

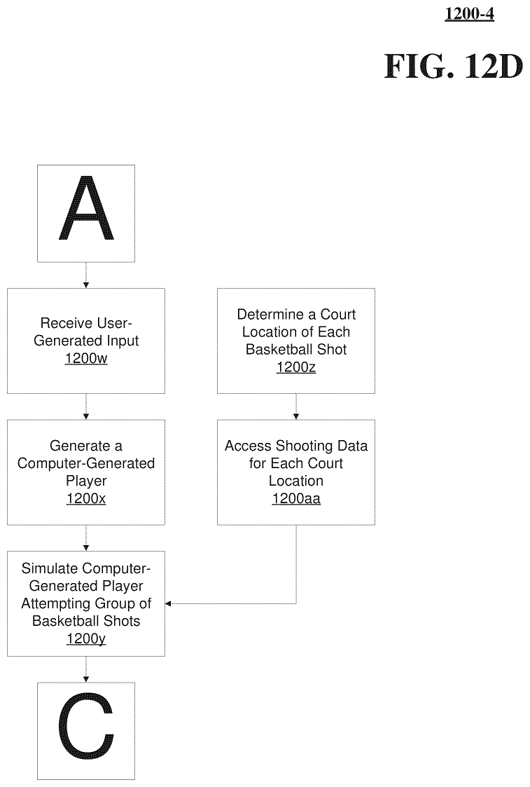

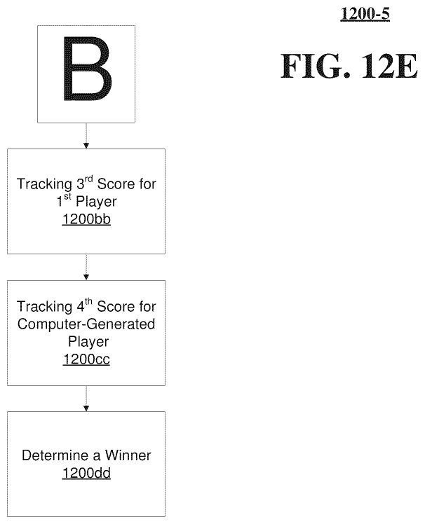

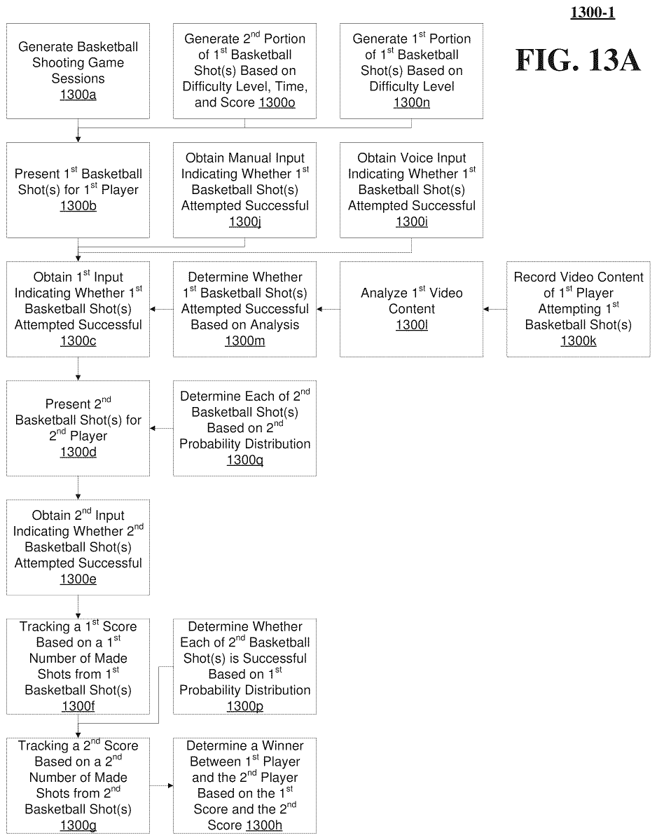

FIG. 12A, FIG. 12B, FIG. 12C, FIG. 12D, FIG. 12E, and FIG. 13A depicts an illustrative embodiment of a method in accordance with various aspects described herein.

Referring to FIG. 12A, one or more embodiments include method 1200. Method 1200 may be useful for operating a gamified basketball shooting training system on a communication device and for generating shooting metrics and instructive biomechanics video content for a first player. In some embodiments, method 1200 may be performed by the smartphone, including the smartphone's processing system and memory executing a group of software applications that include an AI software application. In further embodiments, one or more operations of method 1200 may be performed by the server 1100g of FIG. 11A in communication with the smartphone 1100d over the network 1100f, with data stored in or retrieved from the database 1100h, and with outputs (e.g., prompts, metrics, instructive content) presented on the smartphone 1100d. Thus, the method 1200 can be implemented by a communication device that can be, for example, a smartphone and/or a server.

At block 1200a, in one or more embodiments, the communication device can generate basketball shooting game sessions, the method 1200 involves generating, by a group of software applications, a group of basketball shooting game sessions on the communication device. In some embodiments, block 1200a broadly includes instantiating a series of sessions that each correspond to a simulated “game” experience used for real-world shot practice while tracking results. For example, the group of basketball shooting game sessions can comprise a plurality of sessions organized as a season of games, including tracking per-game outcomes and season progress.

At block 1200b, in one or more embodiments, the communication device can present a group of basketball shots for a first player, the method 1200 involves presenting, by the group of software applications, during each game session, the group of basketball shots for the first player to attempt on a display associated with the communication device. In some embodiments, block 1200b broadly includes generating and outputting shot prompts that instruct the first player what shot to take next. For example, the shot prompts can be selected from a tiered shot library based on difficulty (e.g., rookie/all-star/MVP), position (e.g., guard/forward), time/score context, or other game logic, and can be presented as on-screen text, graphics (e.g., a shot chart), and/or audio prompts.

At block 1200c, in one or more embodiments, the communication device can record video content of the first player attempting each of or a portion of the group of basketball shots, the method 1200 involves recording, by a camera associated with the communication device, video content of the first player attempting each of the group of basketball shots resulting in first current recorded video content. In some embodiments, block 1200c broadly includes capturing continuous or segmented video during the session so that each prompted shot attempt is associated with corresponding recorded frames. For example, the smartphone 1100d camera can record a clip per attempt, or record a longer stream and segment the stream into per-shot clips, optionally time-aligned with prompts presented at block 1200b.

At block 1200d, in one or more embodiments, the communication device can analyze a first current recorded content, the method 1200 involves analyzing, by an artificial intelligence (AI) software application, the first current recorded video content resulting in a first analysis. The group of software applications can comprise the AI software application. In some embodiments, block 1200d broadly includes applying computer vision and/or ML models to the recorded video content to extract shot-relevant events and measurements. For example, the AI software application can detect the player, identify a shot attempt window, classify shot type (e.g., free throw/two pointer/three pointer), and/or detect shot outcome (make/miss) automatically, with a manual fallback for outcome entry if automatic detection is unavailable or uncertain.

At block 1200e, in one or more embodiments, the communication device can determine a first group of shooting metrics, the method 1200 involves, based on the first analysis, determining, by the AI software application, the first group of shooting metrics associated with each of the group of basketball shots. In some embodiments, block 1200e broadly includes calculating per-shot and aggregate metrics for the first player for the session and/or across the season. For example, the first group of shooting metrics can include makes, misses, points, field-goal percentage, two-point percentage, three-point percentage, free-throw percentage, streak measures, and/or shot-type breakdowns; and can be stored locally on the smartphone 1100d and/or in the database 1100h via the server 1100g.

At block 1200f, in one or more embodiments, the communication device can present the first group of shooting metrics, the method 1200 involves presenting, by the group of software applications, the first group of shooting metrics associated with each of the group of basketball shots on the display of the communication device. In some embodiments, block 1200f broadly includes rendering a results interface that communicates performance outcomes to the player in a game-like format. For example, the smartphone 1100d can display per-shot results, session totals, percentages, and/or a shot chart, and can provide audio commentary or score updates synchronized with the metrics.

At block 1200g, in one or more embodiments, the communication device can analyze previously recorded video content of the first player, the method 1200 involves analyzing, by the AI software application, previously recorded video content associated with the first player during, for example, a practice session a previous competitive game (e.g., high school game). In some embodiments, block 1200g broadly includes retrieving and processing prior practice or gameplay video of the same player to establish a reference set of motion patterns and/or successful attempts. For example, the previously recorded video content can be stored in the database 1100h and retrieved by the server 1100g and/or smartphone 1100d for comparative analysis.

At block 1200h, in one or more embodiments, the communication device can determine a portion of previously recorded content is instructive, the method 1200 involves determining, by the AI software application, a portion of the previously recorded content that is instructive with regard to biomechanics of the first player shooting a basketball resulting in first instructive video content. In some embodiments, block 1200h broadly includes selecting segments of prior video that exemplify biomechanical patterns correlated with higher success for that same player. For example, the AI software application can identify prior clips where the player's release timing, jump direction, landing stance, guide-hand behavior, or other biomechanical characteristics align with the player's higher make rates.

At block 1200i, in one or more embodiments, the communication device can present the first instructive video content, the method 1200 involves presenting, by the AI software application, the first instructive video content on the display of the communication device. In some embodiments, block 1200i broadly includes rendering the selected “instructive” portion(s) for review as a coaching aid. For example, the smartphone 1100d can present the first instructive video content as a clip, looped snippet, or annotated playback that highlights the biomechanics that contributed to prior success.

At block 1200j, in one or more embodiments, the communication device can analyze first current recorded video content of the first player, the method 1200 involves analyzing, by the AI software application, the first current recorded video content associated with the first player. In some embodiments, block 1200j broadly includes identifying a current-session segment suitable for instruction, such as a clip illustrating a biomechanical deviation associated with reduced success or inconsistency. For example, the AI software application can locate a current clip where the player's landing stance is inconsistent, the release angle differs from prior successful attempts, or the pre-shot movement differs from a historically successful pattern.

At block 1200k, in one or more embodiments, the communication device can determine a portion of first current recorded content is instructive, the method 1200 involves determining, by the AI software application, a portion of the first current recorded content that is instructive with regard to biomechanics of the first player shooting the basketball resulting in second instructive video content. In some embodiments, block 1200k broadly includes selecting a representative current-session segment for comparison against the first instructive video content. For example, the second instructive video content can include a clip of a missed attempt, a low-quality attempt, or a clip where the player deviates from a previously successful biomechanical profile.

At block 1200l, in one or more embodiments, the communication device can present a second instructive video content, the method 1200 involves presenting, by the AI software application, the second instructive video content on the display of the communication device. In some embodiments, block 1200l broadly includes presenting the current-session instructive segment with optional markings, overlays, or cues. For example, the smartphone 1100d can display the second instructive video content with pose skeleton overlays, highlighted joints, or textual cues indicating a biomechanical difference from prior success clips.

At block 1200m, in one or more embodiments, the communication device can present the first instructive video content and the second instructive video content simultaneously, the method 1200 involves presenting the first instructive video content and the second instructive video content simultaneously. In some embodiments, block 1200m broadly includes arranging both instructive video contents so the player can compare “successful form” versus “current form” in a single view. For example, the smartphone 1100d can play the first instructive video content side-by-side with the second instructive video content in synchronized playback.

At block 1200n, in one or more embodiments, the communication device can present the first instructive video content and the second instructive video content overlaid on one another, the method 1200 involves presenting the first instructive video content and the second instructive video content overlaid on one another. In some embodiments, block 1200n broadly includes aligning the two instructive segments (e.g., by key frames such as gather, release, and landing) and overlaying one on the other to visually reveal biomechanical differences. For example, the overlay can include semi-transparent blending of the two video contents, or overlaying skeletal pose models from the first instructive video content onto frames of the second instructive video content, to emphasize differences in jump direction, landing stance, release timing, or other mechanics.

While for purposes of simplicity of explanation, the respective processes are shown and described as a series of blocks in FIG. 12A, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methods described herein.

Referring to FIG. 12B depicts an illustrative embodiment of a method in accordance with various aspects described herein. Method 1200 (continued in FIG. 12B) may be useful for operating a gamified basketball shooting training system in a two-player configuration. In some embodiments, the method of FIG. 12B may be performed by the smartphone 1100d executing a group of software applications as described herein, including presenting shot prompts and recording video via a camera of the smartphone 1100d. In further embodiments, one or more operations may be performed by the server 1100g in communication with the smartphone 1100d over the network 1100f, with player profiles, shot libraries, and session data stored in and/or retrieved from the database 1100h, and with results presented on the smartphone 1100d. Thus, the method 1200 can be performed by a communication device comprising one of the smartphone and/or the server.

At block 1200o, in one or more embodiments, the communication device can present a group of basketball shots for a second player, the method involves presenting, by the group of software applications during each of the group of basketball shooting game sessions, the group of basketball shots for the second player to attempt on the display associated with the communication device. The second player is associated with each of the group of basketball shooting game sessions. In some embodiments, block 1200o broadly includes generating and displaying shot prompts for the second player in a manner analogous to the first player prompts. For example, the smartphone 1100d can display on-screen text and/or a shot chart indicating a particular shot type and location for the second player to attempt, and can optionally output audible prompts. In some embodiments, the presented shots for the second player can be the same sequence as presented to the first player, can be an alternating sequence (first player prompt then second player prompt), or can be generated based on a selected difficulty level and game logic.

At block 1200p, in one or more embodiments, the communication device can record video content of the second player attempting the group of basketball shots, the method involves recording, by the camera associated with the communication device, video content of the second player attempting each of the group of basketball shots resulting in second current recorded video content. In some embodiments, block 1200p broadly includes capturing video of each prompted shot attempt by the second player such that the recorded content can be analyzed for shooting performance. For example, the smartphone 1100d camera can record per-attempt clips or a continuous stream that is segmented into shot-attempt segments corresponding to the prompts presented at block 1200o.

At block 1200q, in one or more embodiments, the communication device can analyze the second current recorded content, the method involves analyzing, by an AI software application, the second current recorded video content resulting in a second analysis. In some embodiments, block 1200q broadly includes applying computer vision and/or ML models to the second current recorded video content to detect shot attempts and determine shot outcomes and other characteristics. For example, the AI software application can determine whether the second player's prompted shot was a make or a miss, classify the shot type (e.g., free throw, two-point, three-point), and/or associate the attempt with a particular shot prompt presented at block 1200o.

At block 1200r, in one or more embodiments, the communication device can determine a group of second shooting metrics, the method involves, based on the second analysis, determining, by the AI software application, a second group of shooting metrics associated with each of the group of basketball shots. In some embodiments, block 1200r broadly includes computing per-shot and aggregate performance statistics for the second player for a session and/or across multiple sessions. For example, the second group of shooting metrics can include makes, misses, points, field-goal percentage, two-point percentage, three-point percentage, and free-throw percentage, and can be stored locally by the smartphone 1100d and/or stored remotely via the server 1100g in the database 1100h.

At block 1200s, in one or more embodiments, the communication device can present the second group of shooting metrics, the method involves presenting, by the group of software applications, the second group of shooting metrics associated with each of the group of basketball shots on the display of the communication device. In some embodiments, block 1200s broadly includes rendering the second player's metrics in a results interface, optionally alongside the first player's metrics, to support head-to-head gameplay. For example, the smartphone 1100d can display the second player's score and shooting percentages in a post-session screen or in real-time during gameplay, and can also provide audio feedback such as score updates.

While for purposes of simplicity of explanation, the respective processes are shown and described as a series of blocks in FIG. 12B, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methods described herein.

Referring to FIG. 12C, one or more embodiments depict an illustrative embodiment of method 1200 in accordance with various aspects described herein. Method 1200 (continued in FIG. 12C) may be useful for tracking scores for two players during a basketball shooting game session and determining a winner. In some embodiments, the method of FIG. 12C may be performed by the smartphone 1100d executing a group of software applications as described herein, including a gamification software application and, in some embodiments, an AI software application. In further embodiments, one or more operations may be performed by the server 1100g in communication with the smartphone 1100d over the network 1100f, with scoring data and session data stored in and/or retrieved from the database 1100h, and with results presented on the smartphone 1100d (e.g., as shown in the results interface of FIG. 11G). Thus, the method of FIG. 12C can be implemented by the communication device, including the processing system and memory of the smartphone 1100d and/or the server 1100g.

At block 1200t, in one or more embodiments, the communication device can track a first score for the first player, the method involves tracking, by the group of software applications, a first score based on a first number of made shots from the group of basketball shots associated with the first player for each of the group of basketball shooting game sessions. In some embodiments, block 1200t broadly includes incrementing, updating, or otherwise maintaining a score value for the first player based on shot outcomes attributed to the first player during gameplay. For example, the smartphone 1100d can update the first score in response to a make determination generated by the AI software application from recorded video content (e.g., as analyzed in connection with FIG. 12A), and/or in response to a manual make/miss input entered by a user when automatic detection is unavailable.

At block 1200u, in one or more embodiments, the communication device can track a second score for the second player, the method involves tracking, by the group of software applications, a second score based on a second number of made shots from the group of basketball shots associated with the second player for each of the group of basketball shooting game sessions. In some embodiments, block 1200u broadly includes incrementing, updating, or otherwise maintaining a score value for the second player based on shot outcomes attributed to the second player during gameplay. For example, in a two-player mode, the smartphone 1100d can track the second score based on makes detected from video content of the second player (e.g., as analyzed in connection with FIG. 12B), and can store or synchronize the tracked score to the server 1100g and database 1100h for persistence across sessions.

At block 1200v, in one or more embodiments, the communication device can determine a winner, the method involves determining, by the group of software applications, a winner between the first player and the second player for each of the basketball shooting game sessions. In some embodiments, block 1200v broadly includes comparing the first score and the second score to determine an outcome of the game session and generating a win/loss indication. For example, the smartphone 1100d can determine the winner based on which of the first score and the second score is greater at an end-of-session condition (e.g., completion of a predetermined number of prompted shots, expiration of a simulated game clock, or completion of a quarter-based session), and can present the outcome on a results screen along with shooting metrics for each player.

While for purposes of simplicity of explanation, the respective processes are shown and described as a series of blocks in FIG. 12C, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methods described herein.

Referring to FIG. 12D, one or more embodiments, depict an illustrative embodiment of method 1200 in accordance with various aspects described herein. Method 1200 (continued in FIG. 12D) may be useful for enabling a one-player gamified basketball shooting game session against a computer-generated player, including receiving user-generated input, generating the computer-generated player, and simulating the computer-generated player attempting the group of basketball shots. In some embodiments, the method of FIG. 12D may be performed by the smartphone 1100d executing a group of software applications as described herein, including a gamification software application and an AI software application. In further embodiments, one or more operations may be performed by the server 1100g in communication with the smartphone 1100d over the network 1100f, with shot libraries, opponent parameters, and real-life player shooting data stored in and/or retrieved from the database 1100h, and with simulation results and game status presented on the smartphone 1100d. Thus, method 1200 can be performed by a communication device comprising a smartphone and/or a server.

At block 1200w, in one or more embodiments, the communication device can receive user-generated input, the method involves receiving user-generated input associated the first player. In some embodiments, block 1200w broadly includes receiving, by the smartphone 1100d, user selections that configure an opponent experience for a basketball shooting game session. For example, the user-generated input can be entered via a graphical user interface on the smartphone 1100d and can indicate that the first player will play against a computer-generated player, and/or can indicate parameters used to create or configure the computer-generated player, such as a selected difficulty level, a competitive mode, or a selection of a real-life player to emulate.

At block 1200x, in one or more embodiments, the communication deice can generate a computer-generated player, the method involves generating, by the group of software applications, a computer-generated player for each of the group of basketball shooting game sessions. In some embodiments, block 1200x broadly includes creating a virtual opponent entity (e.g., an opponent profile) that will be used by the gamification software application to compete with the first player during the session. For example, the computer-generated player can be generated as a generalized opponent corresponding to a selected level (e.g., rookie, all-star, MVP), and/or can be generated to emulate a specific real-life player selected by the user-generated input.

In some embodiments, the user-generated input received at block 1200w indicates a level associated with the computer-generated player, and generating the computer-generated player at block 1200x comprises generating, by the group of software applications, the computer-generated player based on the level. For example, the level can set scoring probability, shot-type tendencies, shot selection frequencies, or other opponent behavior parameters used in the simulation.

At block 1200y, in one or more embodiments, the communication device can simulate computer-generated player attempting group of basketball shots, the method involves simulating, by the AI software application, the computer-generated player attempting the group of basketball shots. In some embodiments, block 1200y broadly includes executing a simulation routine that produces opponent “attempt” outcomes (e.g., makes/misses and resulting scoring) that can be used to update a scoreboard and determine a winner at the end of a game session. For example, the AI software application can simulate the computer-generated player taking the same prompted shots as the first player, a corresponding set of shots mapped to similar difficulty/locations, or a set of shots generated to match game context (e.g., clock, score differential).

In some embodiments, when the user-generated input indicates a level associated with the computer-generated player, simulating at block 1200y comprises simulating, by the AI software application, the computer-generated player attempting the group of basketball shots based on the level. For example, a higher level can correspond to a higher make probability and/or different shot selection behavior (e.g., more three-point attempts), while a lower level can correspond to a lower make probability and/or simpler shot selection behavior.

At block 1200z, in one or more embodiments, the communication device can determine a court location of each basketball shot, the method involves determining, by the AI software application, a court location of each of the group of basketball shots resulting in a group of court locations. In some embodiments, block 1200z broadly includes mapping each prompted shot to a corresponding region or coordinate on a shot chart (e.g., left corner three, right wing, top of the key, elbow, block, free throw line). For example, the AI software application can derive the court location directly from the shot prompt metadata stored in a shot library and/or database, or from a shot chart representation presented to the first player.

At block 1200aa, in one or more embodiments, the communication device can access shooting data for each court location, the method involves accessing, by the AI software application, shooting data for the real-life player for each of the group of court locations. In some embodiments, block 1200aa broadly includes retrieving historical shooting percentages, attempt distributions, and/or other real-life player performance statistics indexed by court region. For example, the AI software application can access the shooting data from the database 1100h via server 1100g, and use the retrieved shooting data as probability inputs to the simulation such that the computer-generated player makes or misses shots in a manner that emulates the real-life player at the corresponding court locations. In some embodiments, simulating, by the AI software application, the computer-generated player attempting the group of basketball shots can comprise simulating, by the AI software application, the computer-generated player attempting the group of basketball shots based on the shooting data for each court location of the real-life player associated with the computer-generated player. In some embodiments, shooting data for a real-life player to be used for the computer-generated player can be accessed based on the selected difficulty level. For example, if rookie mode is selected, shooting data associated with the rookie year of the real-life player can be accessed. If all-star mode is selected, shooting data associated with one or more all star years of the real-life player can be accessed. If MVP mode is selected, shooting data associated with one or more MVP years of the real-life player can be accessed.

While for purposes of simplicity of explanation, the respective processes are shown and described as a series of blocks in FIG. 12D, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methods described herein.

Referring to FIG. 12E, one or more embodiments depict an illustrative embodiment of a method 1200 in accordance with various aspects described herein. Method 1200 (continued in FIG. 12E) may be useful for tracking scores for a first player and a computer-generated player during a basketball shooting game session and determining a winner. In some embodiments, the method of FIG. 12E may be performed by the smartphone 1100d executing a group of software applications as described herein, including a gamification software application and, in some embodiments, an AI software application. In further embodiments, one or more operations may be performed by the server 1100g in communication with the smartphone 1100d over the network 1100f, with scoring data, opponent parameters, and session data stored in and/or retrieved from the database 1100h, and with results presented on the smartphone 1100d (e.g., as shown in the results interface of FIG. 11G). Thus, the method of FIG. 12E can be implemented by the communication device described herein, including the processing system and memory of the smartphone 1100d and/or the server 1100g.

At block 1200bb, in one or more embodiments, the communication device can track a third score for the first player, the method involves tracking, by the group of software applications, a third score based on a third number of made shots from the group of basketball shots associated with the first player for each of the group of basketball shooting game sessions. In some embodiments, block 1200bb broadly includes incrementing, updating, or otherwise maintaining a score value for the first player based on made shots attributed to the first player during gameplay against a computer-generated opponent. For example, the smartphone 1100d can update the third score in response to a make determination generated by the AI software application from recorded video content of the first player's shot attempts (e.g., as analyzed in connection with FIG. 12A), and/or in response to manual make/miss input when automatic detection is unavailable.

At block 1200cc, in one or more embodiments, the communication device can track a fourth score for the computer-generated player, the method involves tracking, by the group of software applications, a fourth score based on a fourth number of made shots from the group of basketball shots associated with the computer-generated player for each of the group of basketball shooting game sessions. In some embodiments, block 1200cc broadly includes updating an opponent score value based on simulated makes by the computer-generated player during the same game session. For example, the smartphone 1100d (and/or server 1100g) can update the fourth score based on the results of simulating, by the AI software application, the computer-generated player attempting the group of basketball shots (e.g., as described with respect to FIG. 12D), including simulation outcomes driven by a selected difficulty level and/or real-life player shooting data by court location.

At block 1200dd, in one or more embodiments, the communication device can determine a winner, the method involves determining, by the group of software applications, a winner between the first player and the computer-generated player for each of the basketball shooting game sessions. In some embodiments, block 1200dd broadly includes comparing the third score and the fourth score to determine an outcome of the game session and generating a win/loss indication. For example, the smartphone 1100d can determine the winner based on which of the third score and the fourth score is greater at an end-of-session condition (e.g., completion of a predetermined number of prompted shots, expiration of a simulated game clock, or completion of a quarter-based session), and can present the outcome along with shooting metrics for the first player.

Referring to FIG. 13A, in one or more embodiments, aspects of method 1300-1 can be implemented by one or more communication devices, one or more smartphones, one or more servers, or one or more processing systems, or a combination thereof.

At block 1300a, in one or more embodiments, the method 1300-1 can include a communication device generating, by a group of software applications, a group of basketball game sessions on the communication device.

At block 1300b, in one or more embodiments, the method 1300-1 can include a communication device presenting, by the group of software applications, during each of the group of basketball game sessions, a first group of basketball shots for a first player to attempt on a display associated with the communication device, the first player is associated with each of the group of basketball game sessions.

At block 1300c, in one or more embodiments, the method 1300-1 can include a communication device obtaining, by the group of software applications, first input indicating whether each of first group of basketball shots attempted by the first player is successful.

At block 1300d, in one or more embodiments, the method 1300-1 can include a communication device presenting, by the group of software application, during each of the group of basketball game sessions, a second group of basketball shots for a second player to attempt on the display associated with the communication device, the second player is associated with each of the group of basketball s game sessions.

At block 1300e, in one or more embodiments, the method 1300-1 can include a communication device obtaining, by the group of software applications, second input indicating whether each of second group of basketball shots attempted by the first player is successful.

At block 1300f, in one or more embodiments, the method 1300-1 can include a communication device tracking, by the group of software applications, a first score based on a first number of made shots from the first group of basketball shots associated with the first player for each of the group of basketball game sessions.

At block 1300g, in one or more embodiments, the method 1300-1 can include a communication device tracking, by the group of software applications, a second score based on a second number of made shots from the second group of basketball shots associated with the second player for each of the group of basketball game sessions.

At block 1300h, in one or more embodiments, the method 1300-1 can include a communication device determining, by the group of software applications, a winner between the first player and the second player based in the first score and the second score for each of the basketball game sessions.

In one or more embodiments, the second player comprises a computer-generated player.

At block 1300i, in one or more embodiments, the method 1300-1 can include a communication device obtaining, by the group of software applications, voice input from wireless headphones associated with the first player. In some embodiments, the voice input can be obtained via a speaker associated with the communication device. In further embodiments, the obtaining of the first input indicating whether each of the first group of basketball shots attempted by the first player is successful comprises obtaining, by the group of software applications, voice input from wireless headphones associated with the first player.

At block 1300j, in one or more embodiments, the method 1300-1 can include a communication device obtaining, by the group of software applications, manual input or user-generated input from a user interface associated with the group of software applications. In some embodiments, the obtaining of the input indicating whether each of the first group of basketball shots attempted by the first player is successful comprises obtaining, by the group of software applications, manual input from a user interface associated with the group of software applications.

At block 1300k, in one or more embodiments, the method 1300-1 can include a communication device recording, by a camera associated with communication device, video content of the first player attempting each of the first group of basketball shots resulting in first current video content.

At block 1300l, in one or more embodiments, the method 1300-1 can include a communication device analyzing, by the group of software applications, first current video content resulting in an analysis. This analysis can be performed utilizing image recognition or computer vision on the images associated with the first current video content.

At block 1300m, in one or more embodiments, the method 1300-1 can include a communication device determining, by the group of software applications, whether each of the first group of basketball shots attempted by the first player is successful based on the analysis. In further embodiments, the obtaining of the input indicating whether each of the first group of basketball shots attempted by the first player is successful comprises recording, by a camera associated with communication device, video content of the first player attempting each of the first group of basketball shots resulting in first current video content, analyzing, by the group of software applications, first current video content resulting in an analysis, and determining, by the group of software applications, whether each of the first group of basketball shots attempted by the first player is successful based on the analysis.

At block 1300n, in one or more embodiments, the method 1300-1 can include a communication device generating a first portion of the first group of basketball shots based on a difficulty level. The first portion of the first group of basketball shots is associated with a regular game portion of a basketball game session of the group of basketball game sessions.

At block 1300o, in one or more embodiments, the method 1300-1 can include a communication device generating a second portion of the first group of basketball shots based on a difficulty level, remaining time left in the basketball game session, and a current score. The second portion of the first group of basketball shots is associated with a late game portion of a basketball game session of the group of basketball game sessions.

At block 1300p, in one or more embodiments, the method 1300-1 can include a communication device determining whether each of the second group of basketball shots is successful associated with the second player based on a first probability distribution.

At block 1300p, in one or more embodiments, the method 1300-1 can include a communication device determining each of the second group of basketball shots based second probability distribution.

In one or more embodiments, the first player is at a first location and the second player is at a second location. In such embodiments, each player is associated with its own communication device (e.g., smartphone) and data regarding the basketball game session can be sent to each respective communication device via one or more servers. Further, aspects of method 1300-1 can be implemented be one of communication devices and/or one or more of the servers. In other embodiments, each of the first player and the second player are at a same location.

In one or more embodiments, the AI software application be one of the group of software applications. In some embodiments, the AI software application can be a self-contained executable binary in a single communication device, or can be multiple software executables residing in a smartphone, communication device computing device, server, edge computing device, cloud computing device, or a combination thereof.

While for purposes of simplicity of explanation, the respective processes are shown and described as a series of blocks in FIG. 12A, FIG. 12B, FIG. 12C, FIG. 12D, FIG. 12E, and FIG. 13A, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methods described herein. Further portions of some embodiments can be combined with portions of other embodiments.

In addition, a flow diagram may include a “start” and/or “continue” indication. The “start” and “continue” indications reflect that the steps presented can optionally be incorporated in or otherwise used in conjunction with other routines. In this context, “start” indicates the beginning of the first step presented and may be preceded by other activities not specifically shown. Further, the “continue” indication reflects that the steps presented may be performed multiple times and/or may be succeeded by other activities not specifically shown. Further, while a flow diagram indicates a particular ordering of steps, other orderings are likewise possible provided that the principles of causality are maintained.

As may also be used herein, the term(s) “operably coupled to”, “coupled to”, and/or “coupling” includes direct coupling between items and/or indirect coupling between items via one or more intervening items. Such items and intervening items include, but are not limited to, junctions, communication paths, components, circuit elements, circuits, functional blocks, and/or devices. As an example of indirect coupling, a signal conveyed from a first item to a second item may be modified by one or more intervening items by modifying the form, nature or format of information in a signal, while one or more elements of the information in the signal are nevertheless conveyed in a manner than can be recognized by the second item. In a further example of indirect coupling, an action in a first item can cause a reaction on the second item, as a result of actions and/or reactions in one or more intervening items.

Although specific embodiments have been illustrated and described herein, it should be appreciated that any arrangement which achieves the same or similar purpose may be substituted for the embodiments described or shown by the subject disclosure. The subject disclosure is intended to cover any and all adaptations or variations of various embodiments. Combinations of the above embodiments, and other embodiments not specifically described herein, can be used in the subject disclosure. For instance, one or more features from one or more embodiments can be combined with one or more features of one or more other embodiments. In one or more embodiments, features that are positively recited can also be negatively recited and excluded from the embodiment with or without replacement by another structural and/or functional feature. The steps or functions described with respect to the embodiments of the subject disclosure can be performed in any order. The steps or functions described with respect to the embodiments of the subject disclosure can be performed alone or in combination with other steps or functions of the subject disclosure, as well as from other embodiments or from other steps that have not been described in the subject disclosure. Further, more than or less than all of the features described with respect to an embodiment can also be utilized.

What is claimed is:

1. A communication device. comprising:
a processing system including a processor; and
a memory that stores executable instructions that, when executed by the processing system, facilitate performance of operations, the operations comprising:
generating, by a group of software applications, a group of basketball game sessions on the communication device;
presenting, by the group of software applications, during each of the group of basketball game sessions, a first group of basketball shots for a first player to attempt on a display associated with the communication device, wherein the first player is associated with each of the group of basketball game sessions;
obtaining, by the group of software applications, first input indicating whether each of first group of basketball shots attempted by the first player is successful;
presenting, by the group of software application, during each of the group of basketball game sessions, a second group of basketball shots for a second player to attempt on the display associated with the communication device, wherein the second player is associated with each of the group of basketball s game sessions;
obtaining, by the group of software applications, second input indicating whether each of second group of basketball shots attempted by the first player is successful;
tracking, by the group of software applications, a first score based on a first number of made shots from the first group of basketball shots associated with the first player for each of the group of basketball game sessions;
tracking, by the group of software applications, a second score based on a second number of made shots from the second group of basketball shots associated with the second player for each of the group of basketball game sessions; and
determining, by the group of software applications, a winner between the first player and the second player based in the first score and the second score for each of the basketball game sessions.

2. The communication device of claim 1, wherein the second player comprises a computer-generated player.

3. The communication device of claim 1, wherein the obtaining of the first input indicating whether each of the first group of basketball shots attempted by the first player is successful comprises obtaining, by the group of software applications, voice input from wireless headphones associated with the first player.

4. The communication device of claim 1, wherein the obtaining of the input indicating whether each of the first group of basketball shots attempted by the first player is successful comprises obtaining, by the group of software applications, manual input from a user interface associated with the group of software applications.

5. The communication device of claim 1, wherein the obtaining of the input indicating whether each of the first group of basketball shots attempted by the first player is successful comprises:
recording, by a camera associated with communication device, video content of the first player attempting each of the first group of basketball shots resulting in first current video content;
analyzing, by the group of software applications, first current video content resulting in an analysis; and
determining, by the group of software applications, whether each of the first group of basketball shots attempted by the first player is successful based on the analysis.

6. The communication device of claim 1, wherein the operations comprise:
generating a first portion of the first group of basketball shots based on a difficulty level, wherein the first portion of the first group of basketball shots is associated with a regular game portion of a basketball game session of the group of basketball game sessions; and
generating a second portion of the first group of basketball shots based on a difficulty level, remaining time left in the basketball game session, and a current score, wherein the second portion of the first group of basketball shots is associated with a late game portion of a basketball game session of the group of basketball game sessions.

7. The communication device of claim 1, wherein the operations comprise determining whether each of the second group of basketball shots is successful associated with the second player based on a first probability distribution.

8. The communication device of claim 1, wherein the operations comprise determining each of the second group of basketball shots based second probability distribution.

9. The communication device of claim 1, wherein the first player is at a first location and the second player is at a second location.

10. The communication device of claim 1, wherein each of the first player and the second player are at a same location.

11. A non-transitory, machine-readable storage device, comprising executable instructions that, when executed by a communication device including a processor, facilitate performance of operations comprising:
generating, by a group of software applications, a group of basketball game sessions on the communication device;
presenting, by the group of software applications, during each of the group of basketball game sessions, a first group of basketball shots for a first player to attempt on a display associated with the communication device, wherein the first player is associated with each of the group of basketball game sessions;
obtaining, by the group of software applications, voice input from wireless headphones associated with the first player, wherein the voice input indicates whether each of first group of basketball shots attempted by the first player is successful;
presenting, by the group of software application, during each of the group of basketball game sessions, a second group of basketball shots for a second player to attempt on the display associated with the communication device, wherein the second player is associated with each of the group of basketball game sessions;
obtaining, by the group of software applications, input indicating whether each of second group of basketball shots attempted by the first player is successful;
tracking, by the group of software applications, a first score based on a first number of made shots from the first group of basketball shots associated with the first player for each of the group of basketball game sessions;
tracking, by the group of software applications, a second score based on a second number of made shots from the second group of basketball shots associated with the second player for each of the group of basketball game sessions; and
determining, by the group of software applications, a winner between the first player and the second player for each of the basketball game sessions.

12. The non-transitory, machine-readable storage device of claim 11, wherein the second player comprises a computer-generated player.

13. The non-transitory, machine-readable storage device of claim 11, wherein the operations comprise:
generating a first portion of the first group of basketball shots based on a difficulty level, wherein the first portion of the first group of basketball shots is associated with a regular game portion of a basketball game session of the group of basketball game sessions; and
generating a second portion of the first group of basketball shots based on a difficulty level, remaining time left in the basketball game session, and a current score, wherein the second portion of the first group of basketball shots is associated with a late game portion of a basketball game session of the group of basketball game sessions.

14. The non-transitory, machine-readable storage device of claim 11, wherein the operations comprise determining whether each of the second group of basketball shots is successful associated with the second player based on a first probability distribution.

15. The non-transitory, machine-readable storage device of claim 11, wherein the operations comprise determining each of the second group of basketball shots based second probability distribution.

16. The non-transitory, machine-readable storage device of claim 11, wherein the first player is at a first location and the second player is at a second location.

17. The non-transitory, machine-readable storage device of claim 11, wherein each of the first player and the second player are at a same location.

18. A method, comprising:
generating, by a communication device including a processor, by a group of software applications, a group of basketball game sessions on the communication device;
presenting, by the communication device, by the group of software applications, during each of the group of basketball game sessions, a first group of basketball shots for a first player to attempt on a display associated with the communication device, wherein the first player is associated with each of the group of basketball game sessions;
obtaining, by the communication device, by the group of software applications, manual input from the first player via a user interface associated with the group of software applications, wherein the manual input indicates whether each of first group of basketball shots attempted by the first player is successful;
presenting, by the communication device, by the group of software application, during each of the group of basketball game sessions, a second group of basketball shots for a second player to attempt on the display associated with the communication device, wherein the second player is associated with each of the group of basketball game sessions;
obtaining, by the communication device, by the group of software applications, input indicating whether each of second group of basketball shots attempted by the first player is successful;
tracking, by the communication device, by the group of software applications, a first score based on a first number of made shots from the first group of basketball shots associated with the first player for each of the group of basketball game sessions;
tracking, by the communication device, by the group of software applications, a second score based on a second number of made shots from the second group of basketball shots associated with the second player for each of the group of basketball game sessions; and
determining, by the communication device, by the group of software applications, a winner between the first player and the second player for each of the basketball game sessions.

19. The method of claim 18, wherein the second player comprises a computer-generated player.

20. The method of claim 18, comprising:
generating, by the communication device, a first portion of the first group of basketball shots based on a difficulty level, wherein the first portion of the first group of basketball shots is associated with a regular game portion of a basketball game session of the group of basketball game sessions; and
generating, by the communication device, a second portion of the first group of basketball shots based on a difficulty level, remaining time left in the basketball game session, and a current score, wherein the second portion of the first group of basketball shots is associated with a late game portion of a basketball game session of the group of basketball game sessions.
